(19) United States
(12) Patent Application Publication
(10) Pub. No.: US 2009/0217465 A1
(43) Pub. Date: Sep. 3, 2009

(54) Process for Hair Dyeing Comprising Application of Composite Pigment

(76) Inventors: Christian Cremer, Lörrach (DE); Andreas Mühlebach, Frick (CH); Olof Wallquist, Therwil (CH); Thomas Giesenberg, Oberwil (CH); Beate Fröhling, Grenzach-Wyhlen (DE); Richard Lewis Riggs, Mannheim (DE)

Correspondence Address: JoAnn Villamizar;Ciba Corporation/Patent Department, 540 White Plains Road, P.O. Box 2005, Tarrytown, NY, 10591, US

(21) Appl. No.: 12/084,023

(22) PCT Filed: Oct. 16, 2006
(86) PCT No.: PCT/EP2006/067417

§ 371 Date: Mar. 5, 2009

(30) Foreign Application Priority Data
Oct. 26, 2005 (EP) 05109984.4

(51) Int. Cl. A61K 8/58; A61K 8/02

(52) U.S. Cl. 8/405

(57) ABSTRACT

Disclosed is a process for dyeing of keratin-containing fibers comprising treating the fibers with at least one functionalized particle comprising on the surface an organic chromophore which is bound via a bridge member, wherein the particles are based on SiO₂, Al₂O₃ or mixtures thereof, and the functionalized particles carry a positive charge.

The present invention relates to a process for dyeing of keratin-containing fibers, especially human hair, wherein specifically functionalized particles are used as dyes.

It is known, for example from WO 95/01772 that cationic dyes can be used for the dyeing of keratin-containing fibers. This class of dyes exhibits very brilliant shades. A disadvantage however is their unsatisfactory fastness to hydrolysis and to light, their frequently inadequate stability under reducing or oxidizing conditions, and their frequently unsatisfactory storage stability (see: John F. Corbett: “The Chemistry of Hair-Care Products”, JSCD August 1976, p. 290). Frequently, also skin staining is an issue. In addition, there is a need for dyeing hair with compounds which give easily access to the full spectrum of colors.

The object of the present invention is to provide dyes for the dyeing of keratin-containing fibers that are distinguished by deep dyeing and simultaneously having good fastness properties with respect to washing, light, shampooing and rubbing, which preferably exhibit satisfactory stability under reducing or oxidizing dyeing conditions, and which cause less skin staining.

The subject matter of the present invention is based on the idea to use silica or alumina dioxide nano-, sub-micro- or microparticles surface modified with at least a chemically bonded dye, if additionally required a chemically bonded cationic group and, if required to make them compatible to a solvent, a compatibilizer group. With this approach and by using different coloured dyes, it is possible to synthesize rather homodisperse particles with any colour needed. Furthermore, the cationic charge must not necessarily be part of the dye; the cationic charge can also be introduced by other additional groups which allows to adjust the charge to a desired level.

The present invention therefore relates to a process for dyeing of keratin-containing fibers comprising treating the fibers with at least one functionalized particle comprising on the surface an organic chromophore which is bound via a bridge member, wherein the particles are based on SiO₂, Al₂O₃ or mixtures thereof, and the functionalized particles carry a positive charge.

The functionalized particles comprising a covalently bound organic chromophore carry a positive charge (for example with nitrogen, sulfur or phosphorus as charge-carrying atom). Examples of cationic groups are cationic ammonium, phosphonium or sulfonium groups. It is preferred that the particles comprise a cationic ammonium group. Examples of cationic ammonium groups are those of the formula —N(R₁*)₃, wherein the three radicals R₁* can have the same or different meanings, and R₁* is hydrogen; C₁-C₁₂alkyl which can be interrupted by —O— and can be substituted by hydroxyl or phenyl, and wherein the phenyl radical can be further substituted by C₁-C₈alkyl, C₁-C₈alkoxy or halogen; or phenyl which can be substituted by C₁-C₈alkyl, C₁-C₈alkoxy or halogen. It is preferred that R₁ is hydrogen, C₁-C₁₂alkyl or C₁-C₁₂hydroxyalkyl, more preferably hydrogen or C₁-C₁₂alkyl, especially C₁-C₁₂alkyl.

Examples of cationic phosphonium groups are those of the formula —P(R₁*)₃, wherein the three radicals R₁* can have the same or different meanings, and are as defined above.

Examples of sulfonium groups are those of formula —S(R₁*)₂, wherein the two radicals R₁* can have the same or different meanings, and are as defined above.

In the context of the present invention it is to be understood that the cationic groups can also comprise the corresponding anionic counterions.

Anionic counterions denote, for example, an organic or inorganic anion, such as halide, preferably chloride and fluoride, sulfate, hydrogen sulfate, phosphate, boron tetrafluoride, carbonate, bicarbonate, oxalate or C₁-C₈alkyl sulfate, especially methyl sulfate or ethyl sulfate; anionic counterion also denotes lactate, formate, acetate, propionate or a complex anion, such as the zinc chloride double salt. The anionic counterion is especially a halide, preferably chloride, fluoride or iodide, sulfate, hydrogen sulfate, methyl sulfate, ethyl sulfate, phosphate, formate, acetate or lactate. The anionic counterion is more especially fluoride, chloride, iodide, methyl sulfate, ethyl sulfate, formate or acetate.

As to the organic chromophore the definitions and preferences given in the following for D apply.

Preferred are functionalized particles comprising, covalently bound to an oxygen atom on the surface, a radical of formula

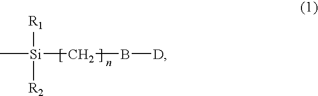

wherein
R₁ and R₂ are independently of each other hydrogen, particle surface-O—, or a substituent,
B is the direct bond or a bridge member,
D is a radical of an organic chromophore, and
n is 1, 2, 3, 4, 5, 6, 7, 8, 9, 10, 11 or 12.

R₁ and R₂ are, for example, independently of each other are hydrogen; C₁-C₂₅alkyl which may be interrupted by —O—, —S— or —N(R₃)—; C₂-C₂₄alkenyl; phenyl; C₇-C₉-phenylalkyl; —OR₅;

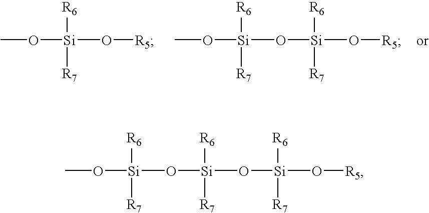

R₅ is hydrogen; C₁-C₂₅alkyl which may be interrupted by —O—, —S— or —N(R₃)—; C₂-C₂₄alkenyl; phenyl; C₇-C₉-phenylalkyl;

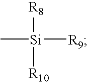

or the particle surface,
R₆ and R₇ independently of each other are hydrogen; C₁-C₂₅alkyl which may be interrupted by —O—, —S— or —N(R₃)—; C₂-C₂₄alkenyl; phenyl; C₇-C₉-phenylalkyl; or —OR₅, and
R₈, R₉ and R₁₀ independently of each other are hydrogen; C₁-C₂₅alkyl which may be interrupted by —O—, —S— or —N(R₃)—; C₂-C₂₄alkenyl; phenyl; or C₇-C₉-phenylalkyl.

R₃ is hydrogen or optionally substituted C₁-C₁₂alkyl. R₃ as alkyl radical may be substituted by the cationic groups mentioned before, especially by a cationic ammonium group. Preferably, R₃ is hydrogen or C₁-C₁₂alkyl, especially hydrogen or C₁-C₄alkyl. A highly preferred meaning for R₃ is hydrogen.

R₁, R₂, R₅, R₆, R₇, R₈, R₉ and R₁₀ as C₁-C₂₅alkyl may be a branched or unbranched radical, for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl, dodecyl, 1,1,3,3,5,5-hexamethylhexyl, tridecyl, tetradecyl, pentadecyl, hexadecyl, heptadecyl, octadecyl, icosyl or docosyl. The alkyl radicals may be uninterrupted or be interrupted by —O—, —S— or —N(R₃)—. Alkyl radicals like C₂-C₂₅alkyl, especially C₃-C₂₅alkyl, which are interrupted by —O— or —S— are, for example, CH₃—O—CH₂CH₂—, CH₃—S—CH₂CH₂—, CH₃—O—CH₂CH₂—O—CH₂CH₂—, CH₃—O—CH₂CH₂—O—CH₂CH₂—, CH₃—(O—CH₂CH₂—)₂O—CH₂CH₂—, CH₃—(O—CH₂CH₂—)₃O—CH₂CH₂— or CH₃—(O—CH₂CH₂—)₄O—CH₂CH₂—.

Preferred is C₁-C₁₂alkyl, especially C₁-C₈alkyl, which alkyl radicals may be uninterrupted or be interrupted by —O—.

R₁, R₂, R₅, R₆, R₇, R₈, R₉ and R₁₀ as alkenyl having 2 to 24 carbon atoms may be a branched or unbranched radical such as, for example, vinyl, propenyl, 2-butenyl, 3-butenyl, isobutenyl, n-2,4-pentadienyl, 3-methyl-2-butenyl, n-2-octenyl, n-2-dodecenyl, iso-dodecenyl, oleyl, n-2-octadecenyl or n-4-octadecenyl. Preference is given to alkenyl having 3 to 18, especially 3 to 12, for example 3 to 6, especially 3 to 4 carbon atoms.

R₁, R₂, R₅, R₆, R₇, R₈, R₉ and R₁₀ as C₇-C₉-phenylalkyl are, for example, benzyl, α-methylbenzyl, α,α-dimethylbenzyl or 2-phenylethyl. Preference is given to benzyl.

R₅ is preferably hydrogen, C₁-C₄alkyl, or the particle surface, especially the particle surface, like the Al₂O₃ surface or the SiO₂ surface. A highly preferred meaning for R₅ is the SiO₂ surface.

R₆, R₇, R₈, R₉ and R₁₀ are preferably C₁-C₄alkyl, especially methyl.

Preferably, R₁ and R₂ are —OR₅;

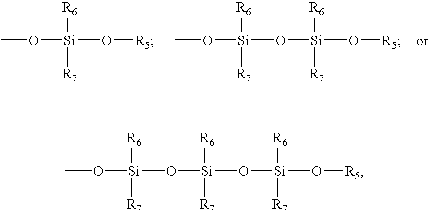

especially a radical of formula —OR₅, wherein for R₅, R₆ and R₇ the above-mentioned meanings and preferences apply.

More preferably, R₁ and R₂ are a radical of formula —OR₅, wherein R₅ is the particle surface, like the Al₂O₃ surface or the SiO₂ surface, especially the SiO₂ surface.

n is preferably 1, 2, 3, 4, 5, 6, 7 or 8, preferably 2, 3 or 4, especially 3.

B is, for example, the direct bond, —O—, —S—, —N(R₃)—, —NH—SO₂—, —NH—CO—, —NH—CO—NH—CO— or C₁-C₂₅alkylene, which alkylene may be bound and/or be interrupted by at least one of the radicals selected from the group consisting of —O—, —S—, —N(R₃)—, —N⁺(R₃)₂—, —CO—, —O—CO—, —CO—O—, —N(R₃)—CO—, —CO—N(R₃)— and phenylene, wherein R₃ is hydrogen or optionally substituted C₁-C₁₂alkyl. The C₁-C₂₅alkylene radical may be unsubstituted or substituted, for example by the cationic groups mentioned before or by hydroxy, preferably by hydroxy. The phenylene radical mentioned above may be unsubstituted or substituted, for example by hydroxyl, halogen, carboxy, sulfonato, amino, acetylamino, mono- or di(C₁-C₈alkyl)amino or the cationic groups mentioned before. R₃ as alkyl radical may be substituted by the cationic groups mentioned before, especially by a cationic ammonium group. Preferably, R₃ is hydrogen or C₁-C₁₂alkyl, especially hydrogen or C₁-C₄alkyl. A highly preferred meaning for R₃ is hydrogen.

Preferably, B is the direct bond, —O—, —S—, —N(R₃)— or a bridge member of formula -A₁-C₁-C₂₅alkylene-A₂-, -A₁-C₁-C₂₅alkylene-phenylene-A₂- or -A₁-phenylene-C₁-C₂₅alkylene-A₂-, wherein the C₁-C₂₅alkylene can be uninterrupted or be interrupted as given above and A₁ and A₂ are the direct bond or radicals as given above. Preferred meanings for A₁ and A₂ are the direct bond, —O—, —S—, —N(R₃)—, —CO—, —O—CO—, —CO—O—, —N(R₃)—CO—, —CO—N(R₃)—, especially —N(R₃)—, —O— or —S—, wherein R₃ is as defined above. Highly preferred meanings for A₁ and A₂ are the direct bond or —N(R₃)—, especially the direct bond or —NH—. As to the C₁-C₂₅alkylene it is preferred that it is uninterrupted or interrupted by at least one of the radicals selected from the group consisting of —O—, —N(R₃)—, —N⁺(R₃)₂—, —CO—, —CO—O—, —CO—N(R₃)— and phenylene, especially —O—, —NH—, —CO—O—, —CO—NH— and phenylene, and more preferably by —CO—O—, —CO—NH— and phenylene. C₁-C₂₅alkylene and phenylene may be substituted as given above, or preferably be unsubstituted. In general, for C₁-C₂₅alkylene radicals C₂-C₂₅alkylene, especially C₂-C₁₆alkylene, is preferred.

More preferably, B is the direct bond, —O—, —S—, —N(R₃)— or a bridge member of formula -A₁-C₁-C₂₅alkylene-A₂-, -A₁-C₁-C₂₅alkylene-phenylene-A₂- or -A₁-phenylene-C₁-C₂₅alkylene-A₂-, wherein

A₁ and A₂ are the direct bond, —O—, —S—, —N(R₃)—, —CO—, —O—CO—, —CO—O—, —N(R₃)—CO— or —CO—N(R₃)—,
the C₁-C₂₅alkylene radical is uninterrupted or interrupted by at least one of the radicals selected from the group consisting of —O—, —S—, —N(R₃)—, —N⁺(R₃)₂—, —CO—, —O—CO—, —CO—O—, —N(R₃)—CO—, —CO—N(R₃)— and phenylene, and
wherein R₃ is as defined above.

Important meanings for B are the direct bond, —O—, —S—, —N(R₃)— or a bridge member of formula -A₁-C₁-C₂₅alkylene-A₂-, -A₁-C₁-C₂₅alkylene-phenylene-A₂- or

-A₁-phenylene-C₁-C₂₅alkylene-A₂-, wherein
A₁ and A₂ are the direct bond —N(R₃)—, —O— or —S—, wherein R₃ is as defined above, and the C₁-C₂₅alkylene radical is uninterrupted or interrupted by at least one of the radicals selected from the group consisting of —O—, —S—, —NH—, —CO—, —O—CO—, —CO—O—, —NH—CO—, —CO—NH— and phenylene.

Very important meanings for B are the direct bond, —O—, —S—, —NH— or a bridge member of formula —NH—C₁-C₂₅alkylene-A₂-, —NH—C₁-C₂₅alkylene-phenylene-A₂- or —NH-phenylene-C₁-C₂₅alkylene-A₂-, wherein

A₂ is the direct bond or —NH—, and
the C₁-C₂₅alkylene radical is uninterrupted or interrupted by at least one of the radicals selected from the group consisting of —CO—O—, —CO—NH— and phenylene.

C₁-C₂₅alkylene and phenylene may be substituted as given above, or preferably be unsubstituted.

D is preferably the radical of an acridine, anthraquinone, azomethine, monoazo, disazo, polyazo, benzodifuranone, coumarin, diketopyrrolopyrrole, dioxazine, diphenylmethane, formazan, indigoid, methine, polymethine, naphthalimide, naphthoquinone, nitroaryl, oxazine, perinone, perylene, phenazine, phthalocyanine, pyrenequinone, quinacridone, quinoneimine, quinophthalone, stilbene, styryl, thiazine, thioxanthene, triarylmethane, xanthene or metal complex dye, and more preferably the radical of an anthraquinone, monoazo, azomethine, methine, polymethine, styryl, triarylmethane or metal complex dye. Highly preferred are radicals of an anthraquinone, monoazo, azomethine or metal complex dye, especially those of a monoazo or azomethine dye.

Preferred radicals D of a monoazo dye are the following:

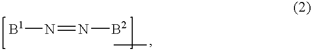

wherein
B¹ and B², independently of each other, are optionally substituted phenyl, naphthyl or a heterocylic group.

As heterocyclic groups corresponding aromatic groups, like imidazole, triazole, thiazole, benzothiazole and pyridine groups, are preferred.

It is especially preferred that B¹ or B² as a heterocyclic group is a radical of an aromatic substituted or unsubstituted heterocyclic group of formula

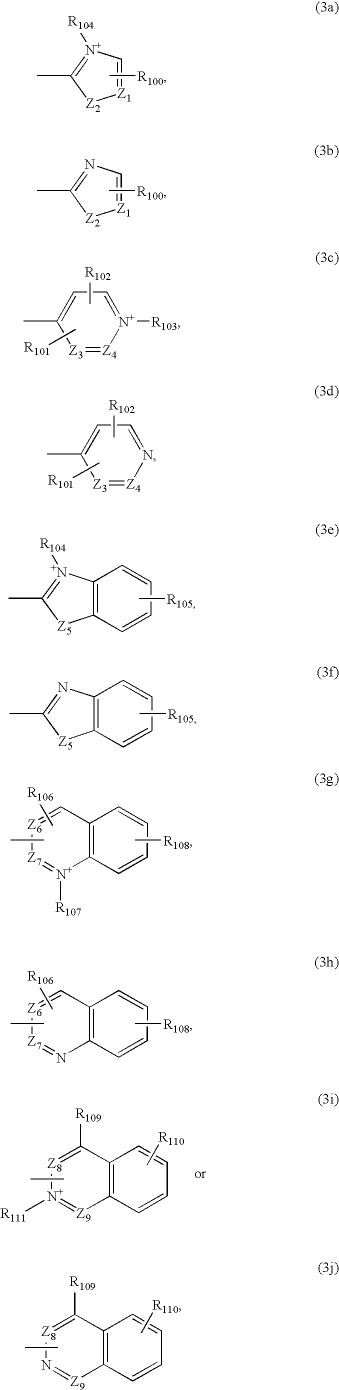

wherein
Z₂ and Z₅ are —O—; —S—; or a radical NR₁₁₂,
Z₁, Z₃, Z₄, Z₆, Z₇, Z₈ and Z₉ are independently from each other N or a radical CR₁₁₃;
R₁₀₀, R₁₀₁, R₁₀₂, R₁₀₅, R₁₀₆, R₁₀₈, R₁₀₉, R₁₁₀ and R₁₁₃ are independently from each other hydrogen; halogen; hydroxy; unsubstituted or substituted C₁-C₁₂alkyl; unsubstituted or substituted phenyl; nitrile; C₂-C₄alkanoylamino; carbamoyl; ureido; sulfonylamino; C₁-C₁₂alkylthio; or a radical of formula —N(R₁₁₄)R₁₁₅, —N(R₁₁₄)(R₁₁₅)R₁₁₆ or —OR₁₁₄;
R₁₀₃, R₁₀₄, R₁₀₇, R₁₁₁ and R₁₁₂ are independently from each other hydrogen; unsubstituted or substituted C₁-C₁₂alkyl; or unsubstituted or substituted phenyl; and
R₁₁₄, R₁₁₅ and R₁₁₆ are independently from each other hydrogen; unsubstituted or substituted C₁-C₁₂alkyl; or unsubstituted or substituted triazinyl or phenyl.

The phenyl and triazinyl radicals mentioned above under formulae (3a) to (3j) and B₁ and B₂ in the meaning as phenyl or naphthyl may be unsubstituted or substituted by, for example, C₁-C₈alkyl; C₁-C₈hydroxyalkyl; C₁-C₈alkoxy; C₁-C₈hydroxyalkoxy; hydroxyl; halogen; or a radical of formula —N(R₁₁₄)R₁₁₅, —N(R₁₁₄)(R₁₁₅)R₁₁₆ or —OR₁₁₄, wherein R₁₁₄, R₁₁₅ and R₁₁₆ are as defined above.

The C₁-C₁₂alkyl radicals mentioned above under formulae (3a) to (3j) may be unsubstituted or substituted by, for example, C₁-C₈alkoxy; C₁-C₈ hydroxyalkoxy; phenyl which can be unsubstituted or substituted as given above; hydroxyl; halogen; or a radical of formula —N(R₁₁₄)R₁₁₅, —N(R₁₁₄)(R₁₁₅)R₁₁₆ or —OR₁₁₄, wherein R₁₁₄, R₁₁₅ and R₁₁₆ are as defined above.

R₁₀₀, R₁₀₁, R₁₀₂, R₁₀₅, R₁₀₆, R₁₀₈, R₁₀₉, R₁₁₀ and R₁₁₃ are preferably independently from each other hydrogen; halogen; hydroxy; C₁-C₁₂alkyl; phenyl; C₂-C₄alkanoylamino; or a radical of formula —N(R₁₁₄)R₁₁₅, —N(R₁₁₄)(R₁₁₅)R₁₁₆ or —OR₁₁₄. Highly preferred are the meanings as hydrogen; halogen; hydroxy; C₁-C₁₂alkyl; or a radical of formula —N(R₁₁₄)R₁₁₅, —N(R₁₁₄)(R₁₁₅)R₁₁₆ or —OR₁₁₄, especially hydrogen.

R₁₀₃, R₁₀₄, R₁₀₇, R₁₁₁ and R₁₁₂ are preferably independently from each other unsubstituted or substituted C₁-C₁₂alkyl; or unsubstituted or substituted phenyl; especially C₁-C₁₂alkyl or phenyl, and more preferably C₁-C₁₂alkyl, especially C₁-C₄alkyl.

As to R₁₁₄, R₁₁₅ and R₁₁₆ it is preferred that for these radicals the definitions and preferences given above R₁, apply.

Z₁, Z₃, Z₄, Z₆, Z₇, Z₈ and Z₉ are preferably a radical CR₁₁₃. As to R₁₁₃ the meaning as hydrogen or C₁-C₁₂alkyl, especially hydrogen, is preferred.

Z₂ is preferably —S— or a radical NR₁₁₂, especially a radical NR₁₁₂. As to R₁₁₂ the meaning as C₁-C₁₂alkyl, especially C₁-C₄alkyl, is preferred.

Z₅ is preferably —S— or a radical NR₁₁₂, especially —S—. As to R₁₁₂ the meaning as C₁-C₁₂alkyl, especially C₁-C₄alkyl, is preferred.

B¹ or B² as a heterocyclic group is more preferably a radical of formula (3a), (3c), (3e), (3g) or (3i), especially a radical of formula (3a), (3c) or (3e). Highly preferred are radicals of formula (3a) or (3c), especially those of formula (3a).

In addition, it is preferred that at least one of B¹ and B² is a phenyl or naphthyl group, especially a phenyl group.

Preferred radicals D of a disazo dye are the following:

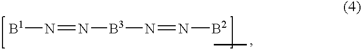

wherein B¹ and B² are as defined above under formula (2) and B³ is phenylene or naphthylene, each of which can be substituted as given above under formula (2) for B¹ and B² in the meaning as phenyl or naphthyl.

Preferred radicals D of an azamethine dye are the following:

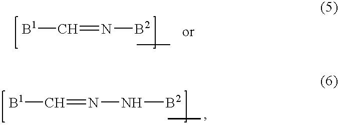

wherein B¹ and B² are as defined above under formula (2). It is preferred that B¹ is a phenyl or naphthyl radical, especially a phenyl radical.

Preferred radicals D of a styryl dye are the following:

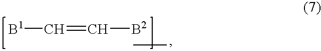

wherein B¹ and B² are as defined above under formula (2). It is preferred that B¹ is a phenyl or naphthyl radical, especially a phenyl radical.

Preferred radicals D of a triarylmethane dye are those of formula:

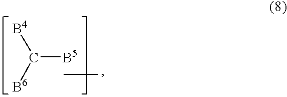

wherein B⁴, B⁵ and B⁶, independently of each other, are phenyl, naphthyl or a heterocyclic group. As to B⁴, B⁵ and B⁶ the definitions and preferences given above under formula (2) for B¹ and B² apply. Preferably, B⁴, B⁵ and B⁶ are corresponding phenyl radicals. It is highly preferred that the triarylmethane dyes of formulae (8) contain at least one group, especially at least three groups, of formula —N(R₁₁₄)R₁₁₅ or —N(R₁₁₄)(R₁₁₅)R₁₁₆, wherein R₁₁₄, R₁₁₅ and R₁₁₆ are as defined above under formula (2).

Preferred radicals D of an anthraquinone dye are the following:

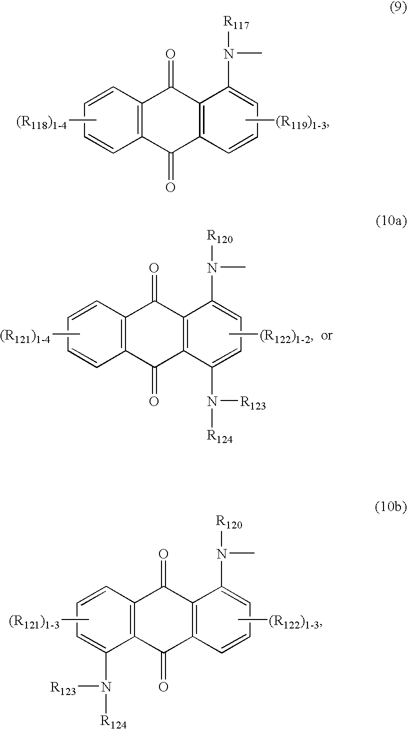

wherein
R₁₁₇, R₁₂₀ and R₁₂₃ are hydrogen; or unsubstituted or substituted C₁-C₁₂alkyl, R₁₁₈, R₁₁₉, R₁₂₁ and R₁₂₂ are hydrogen; unsubstituted or substituted C₁-C₁₂alkyl; C₂-C₄alkanoylamino; halogen; carboxy; sulfonato; ureido; carbamoyl; cyano; nitro; hydroxyl or a radical of formula —N(R₁₁₄)R₁₁₅, —N(R₁₁₄)(R₁₁₅)R₁₁₆ or —OR₁₁₄, wherein R₁₁₄, R₁₁₅ and R₁₁₆ are as defined above; and
R₁₂₄ is hydrogen; unsubstituted or substituted C₁-C₁₂alkyl; or unsubstituted or substituted phenyl.

The phenyl radicals mentioned above under formulae (9), (10a) and (10b) may be unsubstituted or substituted by, for example, C₁-C₈alkyl; C₁-C₈hydroxyalkyl; C₁-C₈alkoxy; C₁-C₈hydroxyalkoxy; hydroxyl; halogen; sulfonato; carboxy; or a radical of formula —N(R₁₁₄)R₁₁₅, —N(R₁₁₄)(R₁₁₅)R₁₁₆ or —OR₁₁₄, wherein R₁₁₄, R₁₁₅ and R₁₁₆ are as defined above.

The C₁-C₁₂alkyl radicals mentioned above under formulae (9), (10a) and (10b) may be unsubstituted or substituted by, for example, C₁-C₈alkoxy; C₁-C₈ hydroxyalkoxy; phenyl which can be unsubstituted or substituted as given above; hydroxyl; halogen; or a radical of formula —N(R₁₁₄)R₁₁₅, —N(R₁₁₄)(R₁₁₅)R₁₁₆ or —OR₁₁₄, wherein R₁₁₄, R₁₁₅ and R₁₁₆ are as defined above.

Preferred radicals D of a metal complex dye are those comprising terpyridine ligands. Preferred metals are iron, especially Fe²⁺.

Preferred terpyridine ligands are those of formula

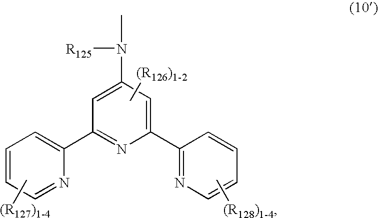

wherein
R₁₂₅ is hydrogen or C₁-C₁₂alkyl;
R₁₂₆, R₁₂₇ and R₁₂₈ are each independently of the others hydrogen; C₁-C₁₂alkyl; C₁-C₁₂alkoxy; hydroxy; phenyl unsubstituted or substituted by C₁-C₈alkyl, C₁-C₈alkoxy, phenyl or by hydroxy; hydrazino; amino; N-mono- or N,N-di-C₁-C₄alkylamino unsubstituted or substituted by hydroxyl in the alkyl moiety; or an unsubstituted or C₁-C₈alkyl-substituted pyrrolidine, piperidine, piperazine, morpholine or azepine ring.

R₁₂₅ is preferably C₁-C₁₂alkyl, more preferably C₁-C₄alkyl. R₁₂₆, R₁₂₇ and R₁₂₈ are preferably hydrogen.

According to a further embodiment of the present invention the functionalized particles can comprise in addition to the radical of formula (1), covalently bound to an oxygen atom on the surface, a radical of formula

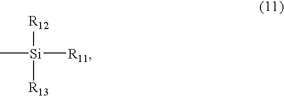

wherein
R₁₂ and R₁₃ have the meanings given above under formula (1) for R₁ and R₂,
R₁₁ is C₁-C₂₅alkyl or C₂-C₂₄alkenyl, each of which is unsubstituted or substituted by amino, mercapto, phenyl or hydroxyl and is uninterrupted or interrupted by —O—, —S—, —N(R₁₄)—, —CO—, —O—CO—, —CO—O—, —N(R₁₄)—CO—, —CO—N(R₁₄)— or phenylene; C₅-C₁₂cycloalkyl; C₅-C₁₂cycloalkenyl; or a polymerizable group or a polymer each of which may be bound via a bridge member, and
R₁₄ is hydrogen or unsubstituted or substituted C₁-C₁₂alkyl, especially hydrogen, C₁-C₁₂alkyl or hydroxyl-substituted C₁-C₁₂alkyl, and more preferably hydrogen or C₁-C₄alkyl.

The radical of formula (11) may, for example, be introduced into the particles in order to compatibilize the particle with a dispersion medium.

As to R₁₂ and R₁₃ the definitions and preferences given herein before for R₁ and R₂ apply.

R₁₄ is preferably hydrogen or methyl, especially hydrogen.

As to R₁₁ in the meaning as C₁-C₂₅alkyl and C₂-C₂₄alkenyl the definitions and preferences given above for R₁, R₂, R₅, R₆, R₇, R₈, R₉ and R₁₀ apply. A preferred definition of R₁₁ is C₂-C₁₂alkyl, especially C₂-C₈alkyl.

R₁₁ as hydroxyl-substituted C₁-C₂₅alkyl is a branched or unbranched radical which contains preferably 1 to 3, in particular 1 or 2, hydroxyl groups, such as, for example, hydroxyethyl, 3-hydroxypropyl, 2-hydroxypropyl, 4-hydroxybutyl, 3-hydroxybutyl, 2-hydroxybutyl, 5-hydroxypentyl, 4-hydroxypentyl, 3-hydroxypentyl, 2-hydroxypentyl, 6-hydroxyhexyl, 5-hydroxyhexyl, 4-hydroxyhexyl, 3-hydroxyhexyl, 2-hydroxyhexyl, 7-hydroxyheptyl, 6-hydroxyheptyl, 5-hydroxyheptyl, 4-hydroxyheptyl, 3-hydroxyheptyl, 2-hydroxyheptyl, 8-hydroxyoctyl, 7-hydroxyoctyl, 6-hydroxyoctyl, 5-hydroxyoctyl, 4-hydroxyoctyl, 3-hydroxyoctyl, 2-hydroxyoctyl, 9-hydroxynonyl, 10-hydroxydecyl, 11-hydroxyundecyl, 12-hydroxydodecyl, 13-hydroxytridecyl, 14-hydroxytetradecyl, 15-hydroxypentadecyl, 16-hydroxyhexadecyl, 17-hydroxyheptadecyl, 18-hydroxyoctadecyl, 20-hydroxyeicosyl or 22-hydroxydocosyl. A preferred definition of R₁₁ is hydroxyl-substituted C₂-C₁₂alkyl, especially hydroxyl-substituted C₄-C₈alkyl.

R₁₁ as alkyl which is interrupted by —O—, —S—, —N(R₁₄)—, —CO—, —O—CO— or —CO—O— is a corresponding C₂-C₂₅alkyl radical, for example, CH₃—O—CH₂CH₂—, CH₃—NH—CH₂CH₂—, CH₃—N(CH₃)—CH₂CH₂—, CH₃—S—CH₂CH₂—, CH₃—O—CH₂CH₂—O—CH₂CH₂—, CH₃—O—CH₂CH₂—O—CH₂CH₂—, CH₃—(O—CH₂CH₂—)₂O—CH₂CH₂—, CH₃—(O—CH₂CH₂—)₃O—CH₂CH₂—, CH₃—(O—CH₂CH₂—)₄O—CH₂CH₂—, CH₃—(O—CH₂CH₂—)₄O—CH₂CH₂—O(CO)—CH₂CH₂—, CH₃CH₂—(O—CH₂CH₂—)₄O—CH₂CH₂—O(CO)—CH₂CH₂— or CH₃—(CH₂)₁₁—O(CO)—CH₂CH₂—.

R₁₁ as alkyl which is substituted by hydroxyl and is interrupted by —O—, —S—, —N(R₁₄)—, —CO—, —O—CO— or —CO—O— is a corresponding C₂-C₂₅alkyl radical, for example, —CH₂—CH(OH)—CH₂—O—CH₃, —CH₂—CH(OH)—CH₂—O—CH₂CH₃, —CH₂—CH(OH)—CH₂—O—CH(CH₃)₂ or —CH₂CH₂—CO—O—CH₂CH₂—O—CO—(CH₂)₅—O—CO—(CH₂)₅—OH.

R₁₁ as alkyl which is substituted by amino-, mercapto- or hydroxyl and is interrupted by —O—, —S—, —N(R₁₄)—, —CO—, —O—CO— or —CO—O— is a corresponding C₂-C₂₅alkyl radical, for example, HO—CH₂CH₂—O—CH₂CH₂—, H₂NCH₂CH₂—NH—CH₂CH₂—, HOCH₂CH₂—NH(CH₃)—CH₂CH₂—, HOCH₂CH₂—S—CH₂CH₂—, H₂NCH₂CH₂—O—CH₂CH₂—O—CH₂CH₂—, HOCH₂CH₂—O—CH₂CH₂—O—CH₂CH₂—, HSCH₂CH₂—(O—CH₂CH₂—)₂O—CH₂CH₂—, H₂NCH₂CH₂—(O—CH₂CH₂—)₃O—CH₂CH₂—, H₂NCH₂CH₂—(O—CH₂CH₂—)₄O—CH₂CH₂—, HSCH₂CH₂—(O—CH₂CH₂—)₄O—CH₂CH₂—O(CO)—CH₂CH₂— or HOCH₂CH₂CH₂CH₂—(O—CH₂CH₂—)₄O—CH₂CH₂—O(CO)—CH₂CH₂—.

R₁₁ as C₅-C₁₂cycloalkyl is, for example, cyclopentyl, cyclohexyl, cycloheptyl, cyclooctyl, cyclononyl, cyclodecyl, cycloundecyl or cyclododecyl. Preference is given to cyclohexyl.

R₁₁ as C₅-C₁₂cycloalkenyl is, for example, cyclopentenyl, cyclohexenyl, cycloheptenyl, cyclooctenyl, cyclononenyl, cyclodecenyl, cycloundecenyl or cyclododecenyl. Preference is given to cyclohexenyl.

R₁₁ as a polymerizable group is, for example,

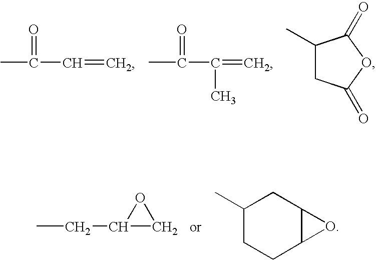

R₁₁ as a polymer is the polymerization product when a polymerizable group, as for example outlined above, is polymerized. In addition, for R₁₁ as a polymer polyorganosiloxanes, like polydimethylsiloxanes, come into consideration. Polydimethylsiloxanes of formula

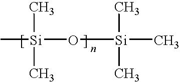

wherein n is a number from 1 to 100, especially 10 to 80, and more preferably 40 to 70, are preferred.

The polymer R₁₁ may be bound via a bridging group. As to this bridging group the definitions and preferences given above for B apply.

R₁₁ is preferably C₁-C₂₅alkyl which is unsubstituted or substituted by hydroxyl, and is uninterrupted or interrupted by —O—, —S—, —N(R₁₄), —CO—, —O—CO—, —CO—O—, —N(R₁₄)—CO— or —CO—N(R₁₄)—, especially by —N(R₁₄)—, —CO—, —O—CO—, —CO—O—, —N(R₁₄)—CO— or —CO—N(R₁₄)—, or R₁₁ is a polyethylene glycol, polypropylene glycol or polyacrylate group which is bound via C₁-C₂₅alkylene, which in turn may be bound and/or be interrupted by at least one of the radicals selected from the group consisting of —O—, —S—, —N(R₁₄), —CO—, —O—CO—, —CO—O—, —N(R₁₄)—CO— or —CO—N(R₁₄)—, especially by —NH—, —CO—, —O—CO—, —CO—O—, —NH—CO— or —CO—NH—.

More preferably R₁₁ is C₁-C₁₂alkyl; C₁-C₁₂alkyl which is substituted by hydroxy; C₁-C₁₂alkyl which is substituted by a polymerizable group, like those given above; C₂-C₂₅alkyl which is interrupted by —NH—, —CO—, —O—CO—, —CO—O—, —NH—CO— or —CO—NH— and which is optionally substituted by hydroxy; or a polyethylene glycol, polypropylene glycol or polyacrylate group which is bound via C₁-C₂₅alkylene, which in turn may be bound and/or be interrupted by at least one of the radicals selected from the group consisting of —NH—, —CO—, —O—CO—, —CO—O—, —NH—CO— or —CO—NH—. It is preferred that the polymer is bound to the alkylene radical via —O—CO— or —CO—O—. As to the alkylene it is preferred that it is bound directly to the Si atom indicated in formula (11). Furthermore, it is preferred that the alkylene is interrupted by at least one of —O—, —S—, —NH—, —CO—, —O—CO—, —CO—O—, —NH—CO— or —CO—NH—, especially by —NH—, —CO—, —O—CO—, —CO—O—, —NH—CO— or —CO—NH—, and more preferably by —NH—, —O—CO—, —CO—O—, —NH—CO— or —CO—NH—.

According to a further embodiment of the present invention the functionalized particles can comprise in addition to the radical of formula (1) or in addition to the radicals of formulae (1) and (11), covalently bound to an oxygen atom on the surface, a radical of formula

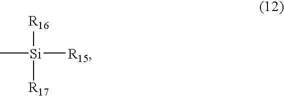

wherein
R₁₆ and R₁₇ have the meanings given above under formula (1) for R₁ and R₂,
R₁₅ is C₁-C₂₅alkyl or C₂-C₂₄alkenyl, each of which is unsubstituted or substituted by amino, mercapto, phenyl or hydroxyl and is uninterrupted or interrupted by —O—, —S—, —N(R₁₈)—, —N⁺(R₁₈)₂—, —CO—, —O—CO—, —CO—O—, —N(R₁₈)—CO—, —CO—N(R₁₈)— or phenylene; C₅-C₁₂cycloalkyl; C₅-C₁₂cycloalkenyl; or a polymerizable group or a polymer each of which may be bound via a bridge member,
R₁₈ is hydrogen or unsubstituted or substituted C₁-C₁₂alkyl, and
wherein R₁₅ or R₁₈ additionally comprise a cationic group, especially a cationic ammonium group, like a group of formula —N(R₁₁₄)(R₁₁₅)R₁₁₆, wherein R₁₁₄, R₁₁₅ and R₁₁₆ are as defined above.

The radical of formula (12) may be introduced into the particles in order to provide the particles with the desired charge. In cases where there exist already radicals providing the charge, like the radical D, the radical of formula (12) may be introduced in order to adjust the charge to a desired level.

As to R₁₆ and R₁₇ the definitions and preferences given herein before for R₁ and R₂ apply.

R₁₈ as alkyl radical may be substituted by the cationic groups mentioned before, especially by a cationic ammonium group, like the group of formula

—N(R₁₁₄)(R₁₁₅)R₁₁₆. Preferably, R₁₈ is hydrogen or C₁-C₁₂alkyl, especially hydrogen or C₁-C₄alkyl. A highly preferred meaning for R₁₈ is hydrogen.

As to R₁₅ the definitions and preferences given herein before for R₁₁ apply. It is to be understood that R₁₅ can be substituted by the cationic groups mentioned above under formula (12). It is preferred that R₁₅ additionally comprises a cationic ammonium group, like the group of formula —N(R₁₁₄)(R₁₁₅)R₁₁₆.

The functionalized particles according to the present invention have preferably a spherical shape.

Preferably, the particles have a mean particle size of 1 to 1000 nm, especially 1 to 600 nm and more preferably 1 to 400 nm. A mean particle size of 1 to 300 nm, especially 1 to 200 nm, is preferred. Very important are particles having a mean particle size of 1 to 100 nm. As a lower limit of the mean particle size 10 nm, especially 20 nm, is preferred. The particle size may, for example, be determined by electron microscopy.

The organic content of the particles according to the present invention is, for example, 5 to 90 percent by weight, especially 20 to 90 percent by weight, and more preferably 40 to 90 percent by weight, based on the total weight of the particle.

Particles are typically silicon dioxide, aluminum oxide, a heterogeneous mixture thereof or silicon aluminum oxide as mixed oxides. The silicon aluminum oxide particles according to the present invention can show silicon contents in between 1 to 99 metal-atom %.

It is preferred that the functionalized particle is a silica (SiO₂) or alumina (Al₂O₃) particle, especially a silica particle.

Unmodified particles, especially such nanoparticles, are commercially available from different suppliers such as Degussa, Hanse Chemie, Nissan Chemicals, Clariant, H. C. Starck, Nanoproducts or Nyacol Nano Technologies as powder or as dispersions. Examples of commercially available silica nanoparticles are Aerosil® from Degussa, Ludox® from DuPont, Snowtex® from Nissan Chemical, Levasil® from Bayer, or Sylysia® from Fuji Silysia Chemical. Examples of commercially available Al₂O₃ nanoparticles are Nyacol® products from Nyacol Nano Technologies Inc., or Disperal® products from Sasol. The artisan is aware of different well-established processes to access particles in different sizes, with different physical properties and with different compositions such as flame-hydrolysis (Aerosil-Process), plasma-process, arc-process and hot-wall reactor-process for gas-phase or solid-phase reactions or ionic-exchange processes and precipitation processes for solution-based reactions. Reference is made to several references describing the detailed processes, such as EP-A-1 236 765, U.S. Pat. No. 5,851,507, U.S. Pat. No. 6,719,821, US-A-2004-178530 or U.S. Pat. No. 2,244,325, WO-A-05/026068, EP-A-1 048 617.

The preparation of the functionalized particles comprising on the surface at least a radical of the formula (1) is preferably carried out by the reaction of corresponding particles (like unfunctionalized silica or alumina particles) with a compound of the formula (1a)

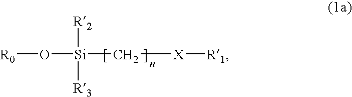

wherein
X is a group like oxygen, sulfur or

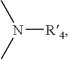

R₀ is C₁-C₂₅alkyl,
R′₁ is hydrogen,

R′₂ and R′₃ independently of each other are hydrogen, C₁-C₂₅alkyl, C₃-C₂₅alkyl which is interrupted by oxygen or sulfur or —N(R₃)—; C₂-C₂₄alkenyl, phenyl, C₇-C₉-phenylalkyl or —OR₁₅,

R′₄ is hydrogen, C₁-C₂₅alkyl or C₃-C₂₅alkyl which is interrupted by oxygen or sulfur or —N(R₃)—;
R′₅ is hydrogen or C₁-C₂₅alkyl, and
n is 1, 2, 3, 4, 5, 6, 7, 8, 9, 10, 11 or 12.

The reaction of the compound of formula (Ia) with the particles can be carried out in analogy to known processes. The reaction can, for example, be carried out in an organic medium or preferably a mixture of water with an organic medium. As organic medium solvents like alcohols, especially methanol or ethanol, can be used. It is preferred to carry out the reaction at temperatures like 20 to 90° C., especially 40 to 60° C. As to the compounds of formula (Ia) it is preferred to use those, wherein at least one of R₀, R′₂ and R′₃ is methoxy or ethoxy, especially wherein R₀, R′₂ and R′₃ are methoxy or ethoxy. It is highly preferred that Ro, R₁₂ and R₁₃ are methoxy. If desired, the products obtained can be redispersed in a suitable medium, like water, ethanol, toluene or xylol.

In a further step the reaction product of the particles with the compound of formula (Ia) can easily be derivatized to obtain particles comprising radicals of the formula (1) by known processes such as for example esterification, amidation, Michael addition or opening of epoxides.

In the following some examples of such reactions are given in general terms:

a) Particles, showing active linkage groups such as —SH or —NH₂ can easily surface modified with educts bearing for instance ester-, epoxy-, carboxy-, carbonyl-, acrylic-, methacrylic-, alkylhalogenide-, alkylsulfate-, anhydride-, terminal double bond-, nitrile- and for instance α,β-unsaturated carbonyl-groups. The chemistry of these substances and the molecular organic syntheses (like nucleophilic substitutions, nucleophilic additions, Michael additions, ring-opening reactions, radical addition, etc.) are well known and can easily be adapted to the solid phase organic chemistry.

b) Particles, showing functional groups on their surfaces, such as ester-, epoxy-, carboxy-, carbonyl, acrylic-, methacrylic-, alkylhalogenide-, alkylsulfate-, anhydride-, terminal double bond-, nitrile- and for instance α,β-unsaturated carbonyl-groups can easily further reacted with educts bearing —SH, —RNH(R=organic group) or —NH₂ groups with the chemical reactions mentioned above under a).

c) In Educts, like dyes, a functional group can be introduced by use of a fluoro-containing dye as starting compound and introduction of the functional group in analogy to the process as described in WO-A-04/076564 (see especially pages 5 to 8 thereof).

d) Educts showing —OH, —RNH(R=organic group) or —NH₂ groups can be activated by using acryloylchloride under basic conditions to generate educt-acrylates (acylation), that can easily be reacted with the particles bearing —SH or —NH₂ groups by using a Michael addition. Other syntheses that are leading to functional groups mentioned in a) and b) are well known.

e) Educts can be functionalized by using reactive alkoxysilanes showing functional groups and mechanisms as mentioned in a), b) or d) and then being grafted onto the particle surface using a state of the art silanisation reaction.

According to an alternative process for the preparation of functionalized particles comprising radicals of formula (1) corresponding unfunctionalized particles, like commercially available silica or Al₂O₃ particles, can be reacted with a compound of the formula (1b)

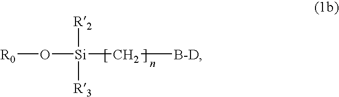

wherein R₀, R′₂ and R′₃ are as defined above under formula (1a) and n, B and D are as defined above under formula (1). By this route the particles comprising a radical of formula (1) can be obtained directly, without further derivatization. The reaction conditions can be chosen as given above for the reaction of the unfunctionalized particles with the compound of formula (1a). The reaction can, for example, be carried out in analogy to the preparation process described in WO-A-03/002652.

The radicals of formulae (11) and (12) can be introduced in analogy to the above preparation processes. These reactions can be carried out simultaneously with the introduction of the radical of formula (1), or stepwise.

As to the preparation methods outlined above it is to be noted that the unfunctionalized particles (like silica or alumina particles) comprise on the surface free hydroxyl groups. These groups are reacted in order to obtain functionalized particles used according to present invention, which can also be described by the following formula

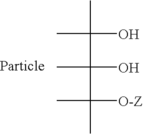

wherein Z is a radical of formula (1) and the vertical line corresponds to the particle surface. In addition, the radicals of formulae (11) and/or (12) may be attached to a hydroxyl group in the same manner as given above for Z.

A further object of the present invention are novel functionalized particles comprising, covalently bound to an oxygen atom on the surface, a radical of formula

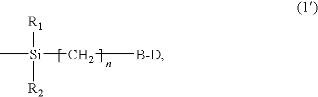

wherein
the particles are based on SiO₂, Al₂O₃ or mixtures thereof,
the functionalized particles carry a positive charge,
R₁ and R₂ are independently of each other hydrogen, particle surface-O—, or a substituent,
B is the direct bond or a bridge member,
n is 1, 2, 3, 4, 5, 6, 7, 8, 9, 10, 11 or 12, and
D is a radical of formula

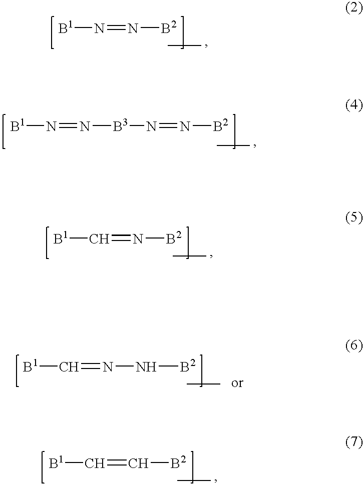

wherein
B³ is unsubstituted or substituted phenylene or naphthylene, and
B¹ and B², independently of each other, are optionally substituted phenyl, naphthyl or a heterocylic group of formula

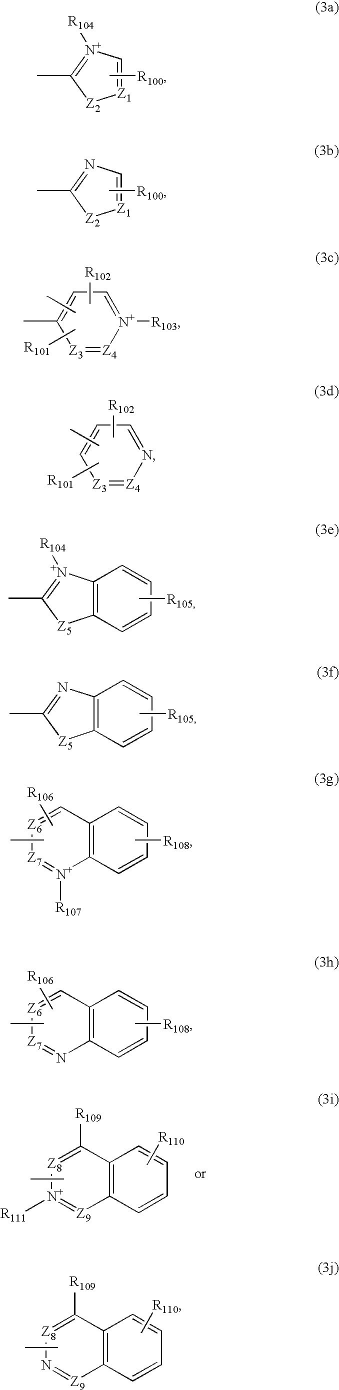

wherein
Z₂ and Z₅ are —O—; —S—; or a radical NR₁₁₂,
Z₁, Z₃, Z₄, Z₆, Z₇, Z₈ and Z₉ are independently from each other N or a radical CR₁₁₃;
R₁₀₀, R₁₀₁, R₁₀₂, R₁₀₅, R₁₀₆, R₁₀₈, R₁₀₉, R₁₁₀ and R₁₁₃ are independently from each other hydrogen; halogen; hydroxy; unsubstituted or substituted C₁-C₁₂alkyl; unsubstituted or substituted phenyl; nitrile; C₂-C₄alkanoylamino; carbamoyl; ureido; sulfonylamino; C₁-C₁₂alkylthio; or a radical of formula —N(R₁₁₄)R₁₁₅, —N(R₁₁₄)(R₁₁₅)R₁₁₆ or —OR₁₁₄;
R₁₀₃, R₁₀₄, R₁₀₇, R₁₁₁ and R₁₁₂ are independently from each other hydrogen; unsubstituted or substituted C₁-C₁₂alkyl; or unsubstituted or substituted phenyl; and
R₁₁₄, R₁₁₅ and R₁₁₆ are independently from each other hydrogen; unsubstituted or substituted C₁-C₁₂alkyl; or unsubstituted or substituted triazinyl or phenyl.

As to the novel functionalized particles comprising a covalently bound radical of formula (1′) the definitions and preferences given above apply. It is preferred that in each of the radicals given for D at least one of the radicals B¹ and B² is a heterocyclic group selected from the formulae (3a) to (3j). Furthermore, it is preferred that the other one of radicals B¹ and B² is unsubstituted or substituted phenyl.

The functionalized particles according to the invention are suitable for dyeing keratin-containing fibers, preferably human hair. The dyeings obtained are distinguished by their depth of shade and their good fastness properties to washing, such as, for example, fastness to light, shampooing and rubbing. The stability, in particular the storage stability of the functionalized particles according to the invention is excellent.

Generally, hair dyeing agents on a synthetic base may be classified into three groups:
temporary dyeing agents
semipermanent dyeing agents, and
permanent dyeing agents.

The multiplicity of shades of the functionalized particles of the invention can be increased by combination with other dyes.

Therefore the functionalized particles of the present invention may be combined with dyes of the same or other classes of dyes, especially with direct dyes, oxidation dyes; dye precursor combinations of a coupler compound as well as a diazotized compound, or a capped diazotized compound; and/or cationic reactive dyes.

Direct dyes are of natural origin or may be prepared synthetically. They are uncharged, cationic or anionic, such as acid dyes.

The functionalized particles of the invention may be used in combination with at least one single direct dye different from the functionalized particles of the invention.

Direct dyes do not require any addition of an oxidizing agent to develop their dyeing effect. Accordingly, the dyeing results are less permanent than those obtained with permanent dyeing compositions. Direct dyes are therefore preferably used for semipermanent hair dyeings.

Examples of direct dyes are described in “Dermatology”, edited by Ch. Culnan, H. Maibach, Verlag Marcel Dekker Inc., New York, Basle, 1986, Vol. 7, Ch. Zviak, The Science of Hair Care, chapter 7, p. 248-250, and in “Europäisches Inventar der Kosmetikrohstoffe”, 1996, published by The European Commission, obtainable in diskette form from the Bundesver-band der deutschen Industrie-und Handelsunternehmen für Arzneimittel, Reformwaren und Körperpflegemittel e.V., Mannheim.

More preferred direct dyes which are useful for the combination with at least one functionalized particle of the invention, especially for semi permanent dyeing, are: 2-amino-3-nitrophenol, 2-amino-4-hydroxyethylamino-anisole sulfate, 2-amino-6-chloro-4-nitrophenol, 2-chloro-5-nitro-N-hydroxyethylene-p-phenylendiamine, 2-hydroxyethyl-picramic acid, 2,6-diamino-3-((pyridine-3-yl)-azo)pyridine, 2-nitro-5-glyceryl-methylaniline, 3-methylamino-4-nitro-phenoxyethanol, 4-amino-2-nitrodiphenyleneamine-2′-carboxilic acid, 6-nitro-1,2,3,4,-tetrahydroquinoxaline, 4-N-ethyl-1,4-bis(2′-hydroxyethylamino-2-nitrobenzene hydrochloride, 1-methyl-3-nitro-4-(2′-hydroxyethyl)-aminobenzene, 3-nitro-p-hydroxyethyl-aminophenol, 4-amino-3-nitrophenol, 4-hydroxypropylamine-3-nitrophenol, hydroxyanthrylaminopropylmethyl morphlino methylsulfate, 4-nitrophenyl-aminoethylurea, 6-nitro-p-toluidine, Acid Blue 62, Acid Blue 9, Acid Red 35, Acid Red 87 (Eosin), Acid Violet 43, Acid Yellow 1, Basic Blue 3, Basic Blue 6, Basic Blue 7, Basic Blue 9, Basic Blue 12, Basic Blue 26, Basic Blue 99, Basic Brown 16, Basic Brown 17, Basic Red 2, Basic Red 22, Basic Red 76, Basic Violet 14, Basic Yellow 57, Basic Yellow 9, Disperse Blue 3, Disperse Orange 3, Disperse Red 17, Disperse Violet 1, Disperse Violet 4, Disperse Black 9, Fast Green FCF, HC Blue 2, HC Blue 7, HC Blue 8, HC Blue 12, HC Orange 1, HC Orange 2, HC Red 1, HC Red 10-11, HC Red 13, HC Red 16, HC Red 3, HC Red BN, HC Red 7, HC Violet 1, HC Violet 2, HC Yellow 2, HC Yellow 5, HC Yellow 5, HC Yellow 6, HC Yellow 7, HC Yellow 9, HC Yellow 12, HC Red 8, hydroxyethyl-2-nitro-p-toluidine, N,N-Bis-(2-Hydroxyethyl)-2-nitro-p-phenylendiamine, HC Violet BS, Picramic Acid, Solvent Green 7.

Furthermore, the functionalized particles of the invention may be combined with at least one cationic azo dye, for example the compounds disclosed in GB-A-2 319 776 as well as the oxazine dyes described in DE-A-299 12 327 and mixtures thereof with the other direct dyes mentioned therein, and even more preferred with cationic dyes such as Basic Yellow 87, Basic Orange 31 or Basic Red 51, or with cationic dyes as described in WO 01/66646, especially example 4, or with cationic dyes as described in WO 02/31056, especially example 6 (compound of formula 106); or the cationic dye of formula (3) as described in EP-A-714,954, or with a yellow cationic dye of formula

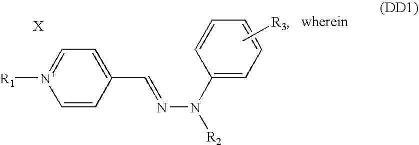

R₁ and R₂ are each independently of the other a C₁-C₈alkyl; or an unsubstituted or substituted benzyl;
R₃ is hydrogen; C₁-C₈alkyl; C₁-C₈alkoxy; cyanide; or halide; preferably hydrogen; and
X⁻ is an anion; and preferably a compound of formula (DD1), wherein
R₁ is methyl; R₂ is benzyl; R₃ is hydrogen; and X— is an anion; or wherein
R₁ is benzyl; R₂ is benzyl; R₃ is hydrogen; and X— is an anion; or wherein
R₁ is benzyl; R₂ is methyl; R₃ is hydrogen; and X— is an anion.

Furthermore, cationic nitroaniline and anthraquinone dyes are useful for a combination with a functionalized particle of the invention, for example the dyes as described in the following patent specifications: U.S. Pat. No. 5,298,029, especially in col 2, I. 33 to col 5, I. 38; U.S. Pat. No. 5,360,930, especially in col 2, I. 38 to col 5, I. 49; U.S. Pat. No. 5,169,403, especially in col 2, I. 30 to col 5, I. 38; U.S. Pat. No. 5,256,823, especially in col 4, I. 23 to col 5, I. 15; U.S. Pat. No. 5,135,543, especially in col 4, I. 24 to col 5, I. 16; EP-A-818 193, especially on p. 2, I. 40 to p. 3, I. 26; U.S. Pat. No. 5,486,629, especially in col 2, I. 34 to col 5, I. 29; and EP-A-758 547, especially on p. 7, I. 48 to p. 8, I. 19.

The functionalized particles of the invention may also be combined with acid dyes, for example the dyes which are known from the international names (Color index), or trade names.

Preferred acid dyes which are useful for the combination with the functionalized particles of the invention are described in U.S. Pat. No. 6,248,314. They include Red Color No. 120, Yellow Color No. 4, Yellow Color No. 5, Red Color No. 201, Red Color No. 227, Orange Color No. 205, Brown Color No. 201, Red Color No. 502, Red Color No. 503, Red Color No. 504, Red Color No. 506, Orange Color No. 402, Yellow Color No. 402, Yellow Color No. 406, Yellow Color No. 407, Red Color No. 213, Red Color No. 214, Red Color No. 3, Red Color No. 104, Red Color No. 105(1), Red Color No. 106, Green Color No. 2, Green Color No. 3, Orange Color No. 207, Yellow Color No. 202(1), Yellow Color No. 202(2), Blue Color No. 202, Blue Color No. 203, Blue Color No. 205, Blue Color No. 2, Yellow Color No. 203, Blue Color No. 201, Green Color No. 201, Blue Color NO. 1, Red Color No. 230(1), Red Color No. 231, Red Color No. 232, Green Color No. 204, Green Color No. 205, Red Color No. 401, Yellow Color No. 403(1), Green Color No. 401, Green Color No. 402, Black Color No. 401 and Purple Color No. 401, especially Black Color No. 401, Purple Color 401, Orange Color No. 205.

These acid dyes may be used either as single component or in any combination thereof.

Hair dye compositions comprising an acid dye are known. They are for example described in “Dermatology”, edited by Ch. Culnan, H. Maibach, Verlag Marcel Dekker Inc., New York, Basle, 1986, Vol. 7, Ch. Zviak, The Science of Hair Care, chapter 7, p. 248-250, especially on p. 253 and 254.

Hair dye compositions which comprise an acid dye have a pH of 2-6, preferably 2-5, more preferably 2.5-4.0.

The functionalized particles of the invention may also readily be used in combination with acid dyes and/or adjuvants, for example
acid dyes and an alkylene carbonate, as described in U.S. Pat. No. 6,248,314, especially in examples 1 and 2;
acid hair dye compositions comprising various kinds of organic solvents represented by benzyl alcohol as a penetrant solvent have good penetrability into hair, as described in Japanese Patent Application Laid-Open Nos. 210023/1986 and 101841/1995;
acid hair dye compositions with a water-soluble polymer or the like to prevent the drooping of the hair dye composition, as described for example in Japanese Patent Application Laid-Open Nos. 87450/1998, 255540/1997 and 245348/1996;
acid hair dye compositions with a water-soluble polymer of aromatic alcohols, lower alkylene carbonates, or the like as described in Japanese Patent Application Laid-Open No. 53970/1998 and Japanese Patent Invention No. 23911/1973.

The functionalized particles of the invention may also be combined with uncharged dyes, for example selected from the group of the nitroanilines, nitrophenylenediamines, nitroaminophenols, anthraquinones, indophenols, phenazines, phenothiazines, bispyrazolons, bispyrazol aza derivatives and methines.

Furthermore, the functionalized particles of the invention may also be used in combination with oxidation dye systems.

Oxidation dyes, which, in the initial state, are not dyes but dye precursors are classified according to their chemical properties into developer and coupler compounds.

Suitable oxidation dyes are described for example in
DE 19 959 479, especially in col 2, I. 6 to col 3, I. 11;
“Dermatology”, edited by Ch. Culnan, H. Maibach, Verlag Marcel Dekker Inc., New York, Basle, 1986, Vol. 7, Ch. Zviak, The Science of Hair Care, chapter 8, on p. 264-267 (oxidation dyes).

Preferred developer compounds are for example primary aromatic amines, which are substituted in the para- or ortho-position with a substituted or unsubstituted hydroxy- or amino residue, or diaminopyridine derivatives, heterocyclic hydrazones, 4-aminopyrazol derivatives, 2,4,5,6-tetraminopyrimidine derivatives, or unsaturated aldehydes as described in DE 19 717 224, especially on p. 2, I. 50 to I. 66 and on p. 3 I. 8 to I. 12, or cationic developer compounds as described in WO 00/43367, especially on p., 2 I. 27 to p. 8, I. 24, in particular on p. 9, I. 22 to p. 11, I. 6.

Furthermore, developer compounds in their physiological compatible acid addition salt form, such as hydrochloride or sulfate can be used. Developer compounds, which have aromatic OH radicals are also suitable in their salt form together with a base, such as alkali metal-phenolates.

Preferred developer compounds are disclosed in DE 19959479, p. 2, I. 8-29.

More preferred developer compounds are p-phenylendiamine, p-toluoylendiamine, p-, m-o-aminophenol, N,N-bis-(2-hydroxyethyl)-p-phenylenediamine sulfate, 2-amino-4-hydroxyethylaminoanisole sulfate, hydroxyethyl-3,4-methylenedioxyanil., 1-(2′-hydroxyethyl)-2,5-diaminobenzene, 2,6-dimethoxy-3,5-diamino-pyridine, hydroxypropyl-bis-(N-hydroxy-ethyl-p-phenylenediamine) hydrochloride, hydroxyethyl-p-phenylenediamine sulfate, 4-amino-3-methylphenol, 4-methylaminophenol sulfate, 2-aminomethyl-4-aminophenol, 4,5-di-amino-1-(2-hydroxyethyl)-1H-pyrazol, 4-amino-m-cresol, 6-amino-m-cresol, 5-amino-6-chloro-cresol, 2,4,5,6-tetraminopyrimidine, 2-hydroxy-4,5,6-triaminopyrimidine or 4-hydroxy-2,5,6-triaminopyrimidine sulfate.

Preferred coupler compounds are m-phenylendiamine derivatives, naphthole, resorcine and resorcine derivatives, pyrazolone and m-aminophenol derivatives, and most preferably the coupler compounds disclosed in DE 19959479, p. 1, I. 33 to p. 3, I. 11.

The functionalized particles of the invention may also be used together with unsaturated aldehydes as disclosed in DE 19 717 224 (p. 2, I. 50 to I. 66 and on p. 3 I. 8 to I. 12) which may be used as direct dyes or, alternatively together with oxidation dye precursors.

Further preferred for a combination with the functionalized particles of the invention are the following oxidation dye precursors:
the developer/-coupler combination 2,4,5,6-tetraminopyrimidine and 2-methylresorcine for assessing of red shades;
p-toluenediamine and 4-amino-2-hydroxytoluene for assessing of blue-violet shades;
p-toluenediamine and 2-amino-4-hydroxyethylaminoanisole for assessing of blue shades;
p-toluenediamine and 2,4-diamino-phenoxyethynol for assessing of blue shades;
methyl-4-aminophenol and 4-amino-2-hydroxytoluene for assessing of orange shades;
p-toluenediamine and resorcine for assessing of brown-green shades;
p-toluenediamine and 1-naphthol for assessing of blue-violet shades, or
p-toluenediamine and 2-methylresorcine for assessing of brown-gold shades.

Furthermore, autooxidizable compounds may be used in combination with the functionalized particles of the invention.

Autooxidizable compounds are aromatic compounds with more than two substituents in the aromatic ring, which have a very low redox potential and will therefore be oxidized when exposed to the air. The dyeings obtained with these compounds are very stable and resistant to shampoo.

Autooxidizable compounds are for example benzene, indole, or indol., especially 5,6-dihydroxyindole or 5,6-dihydroxyindole derivatives as described in WO 99/20234, especially on p. 26, I. 10 to p. 28, I. 15, or in WO 00/28957 on p. 2, third paragraph.

Preferred autooxidizable benzene derivatives are 1,2,4-trihydroxybenzene, 1-methyl-2,4,5-trihydroxybenzene, 2,4-diamino-6-methylphenol, 2-amino-4-methylaminophenol, 2,5-diamino-4-methyl-phenol, 2,6-diamino-4-diethylaminophenol, 2,6-diamino-1,4-dihydroxy-benzene, and the salts of these compounds, which are accessible with acid.

Preferred autooxidizable indole derivatives are 5,6-dihydroxyindole, 2-methyl-5,6-dihydroxy-indole, 3-methyl-5,6-dihydroxyindole, 1-methyl-5,6-dihydroxyindole, 2,3-dimethyl-5,6-dihydroxyindole, 5-methoxy-6-dihydroxyindole, 5-acetoxy-6-hydroixyindole, 5,6-diacetoxyindole, acid of 5,6-dihydroxyindole-2-carbonacid, and the salts of these compounds, which are accessible with acid.

The functionalized particles of the invention may also be used in combination with naturally occurring dyes, such ashenna red, henna neutral, henna black, camomile blossom, sandalwood, black tea, Rhamnus frangula bark, sage, campeche wood, madder root, catechu, sedre and alkanet root. Such dyeings are described, for example, in EP-A-404 868, especially on p. 3, I. 55 to p. 4, I. 9.

Furthermore, the functionalized particles of the invention may also be used in combination with capped diazotised compounds.

Suitable diazotised compounds are for example the compounds of formulae (1)-(4) in WO 2004/019897 (bridging pages 1 and 2) and the corresponding water-soluble coupling components (I)-(IV) as disclosed in the same reference on p. 3ff.

Further preferred dyes or dye combinations which are useful for the combination with the functionalized particles of the invention are described in
(DC-01): WO 95/01772, wherein mixtures of at least two cationic dyes are disclosed, especially p. 2, I. 7 to p. 4, I. 1, preferably p. 4, I. 35 to p. 8, I. 21; formulations p. 11, last §-p. 28, I. 19;
(DC-02): U.S. Pat. No. 6,843,256, wherein cationic dyes are disclosed, especially the compounds of formulae (1), (2), (3) and (4) (col. 1, I. 27-col. 3, I. 20), and preferably the compounds as prepared in the examples 1 to 4 (col. 10, I. 42 to col. 13, I. 37); formulations col. 13, I. 38 to col. 15, I. 8;
(DC-03): EP 970 685, wherein direct dyes are described, especially p. 2, I. 44 to p. 9, I. 56 and preferably p. 9, I. 58 to p. 48, I. 12; processes for dyeing of keratin-containing fibers especially p. 50, I. 15 to 43; formulations p. 50, I. 46 to p. 51, I. 40;
(DC-04): DE-A-19 713 698, wherein direct dyes are described, especially p. 2, I. 61 to p. 3, I. 43; formulations p. 5, I. 26 to 60;
(DC-05): U.S. Pat. No. 6,368,360, wherein direct dyes (col. 4, I. 1 to col. 6, I. 31) and oxidizing agents (col. 6, I. 37-39) are disclosed; formulations col. 7, I. 47 to col. 9, I. 4;
(DC-06): EP 1 166 752, wherein cationic dyes (p. 3, I. 22-p. 4, I. 15) and anionic UV-absorbers (p. 4, I. 27-30) are disclosed; formulations p. 7, I. 50-p. 9, I. 56;
(DC-07): EP 998,908, wherein oxidation dyeings comprising a cationic direct dye and pyrazolo-[1,5-a]-pyrimidines (p. 2, I. 48-p. 4, I. 1) are disclosed; dyeing formulations p. 47, I. 25 to p. 50, I. 29;
(DC-08): FR-2788432, wherein combinations of cationic dyes with Arianors are disclosed, especially p. 53, I. 1 to p. 63, I. 23, more especially p. 51 to 52, most especially Basic Brown 17, Basic brown 16, Basic Red 76 and Basic Red 118, and/or at least one Basic Yellow 57, and/or at least one Basic Blue 99; or combinations of arianoren and/or oxidative dyes, especially p. 2, I. 16 to p. 3, I. 16; dyeing formulations on p. 53, I. 1 to p. 63, I. 23;
(DC-09): DE-A-19 713 698, wherein the combinations of direct dyes and permanent-wave fixing comprising an oxidation agent, an oxidation dye and a direct dye are disclosed; especially p. 4, I. 65 to p. 5, I. 59;
(DC-10): EP 850 638, wherein developer compounds and oxidizing agents are disclosed; especially p. 2, I. 27 to p. 7, I. 46 and preferably p. 7, I. 20 to p. 9, I. 26; dyeing formulations p. 2, I. 3-12 and I. 30 to p. 14, and p. 28, I. 35-p. 30, I. 20; preferably p. 30, I. 25-p. 32, I. 30;
(DC-11): U.S. Pat. No. 6,190,421 wherein extemporaneous mixtures of a composition (A) containing one or more oxidation dye precursors and optionally one or more couplers, of a composition (B), in powder form, containing one or more direct dyes (col. 5, I. 40-col. 7, I. 14), optionally dispersed in an organic pulverulent excipient and/or a mineral pulverulent excipient, and a composition (C) containing one or more oxidizing agents are disclosed; formulations col. 8, I. 60-col. 9, I. 56;
(DC-12): U.S. Pat. No. 6,228,129, wherein a ready-to-use composition comprising at least one oxidation base, at least one cationic direct dye and at least one enzyme of the 2-electron oxidoreductase type in the presence of at least one donor for the said enzyme are disclosed; especially col. 8, I. 17-col. 13, I. 65; dyeing formulations in col. 2, I. 16 to col. 25, I. 55, a multi-compartment dyeing device is described in col. 26, I, 13-24;
(DC-13): WO 99/20235, wherein compositions of at least one cationic dye and at least one nitrated benzene dye with cationic direct dyes and nitro benzene direct dyes are described; on p. 2, I. 1 to p. 7, I. 9, and p. 39, I. 1 to p. 40 I. 11, preferably p. 8, I. 12 to p. 25 I. 6, p. 26, I. 7 to p. 30, I. 15; p. 1, I. 25 to p. 8, I. 5, p. 30, I. 17 to p. 34 I. 25, p. 8, I. 12 to p. 25 I. 6, p. 35, I. 21 to 27, especially on p. 36, I. 1 to p. 37;
(DC-14): WO 99/20234, wherein compositions comprising at least one direct cationic dye and at least one autooxidisable dye, especially benzene, indole and indole derivatives are described, preferably direct dyes on p. 2, I. 19 to p. 26, I. 4, and autooxidisable dyes as disclosed especially on p. 26, I. 10 to p. 28, I. 15; dyeing formulations especially on p. 34, I. 5 to p. 35, I. 18;
(DC-15): EP 850 636, wherein oxidation dyeing compositions comprising at least one direct dye and at least one meta-aminophenol derivative as coupler component and at least one developer compound and an oxidizing agent are disclosed, especially p. 5, I. 41 to p. 7, I. 52, dyeing formulations p. 19, I. 50-p. 22, I. 12;
(DC-16): EP-A-850 637, wherein oxidation dyeing compositions comprising at least one oxidation base selected from para-phenylenediamines and bis(phenyl)alkylenediamines, and the acid-addition salts thereof, at least one coupler selected from meta-diphenols, and the acid-addition salts thereof, at least one cationic direct dye, and at least one oxidizing agent are disclosed, especially p. 6, I. 50 to p. 8, I. 44 are disclosed; dyeing formulations p. 21, I. 30-p. 22, I. 57;
(DC-17): WO 99/48856, wherein oxidation dyeing compositions comprising cationic couplers are disclosed, especially p. 9, I. 16-p. 13, I. 8, and p. 11, I. 20-p. 12, I. 13; dyeing formulations p. 36, I. 7-p. 39, I. 24;
(DC-18): DE 197 172 24, wherein dyeing agents comprising unsaturated aldehydes and coupler compounds and primary and secondary amino group compounds, nitrogen-containing heterocyclic compounds, amino acids, oligopeptides, aromatic hydroxy compounds, and/or at least one CH-active compound are disclosed, p. 3, I. 42-p. 5 I. 25; dyeing formulations p. 8, I. 25-p. 9, I. 61.

In the dye combinations disclosed in the references (DC-01-DC-18) above, the functionalized particles of the invention may be added to the dye combinations or dyeing formulations or may be replaced with at least one functionalized particle of the invention.

The present invention also relates to formulations, which are used for the dyeing of keratin-containing fibers, and most preferably human hair, comprising at least one functionalized particle of the invention.

The functionalized particles of the invention may be incorporated into the formulation in amounts of 0.001-5% b.w. (hereinafter indicated merely by “%”), particularly 0.005-4%, more particularly 0.2-3%, based on the total weight of the formulation.

The formulations may be applied on the keratin-containing fiber, preferably the human hair, in different technical forms.

Technical forms of formulations are for example a solution, especially a thickened aqueous or aqueous alcoholic solution, a cream, foam, shampoo, powder, gel, or emulsion.

Customarily the dyeing compositions are applied to the keratin-containing fiber in an amount of 50 to 100 g.

Preferred forms of formulations are ready-to-use compositions or multi-compartment dyeing devices or ‘kits’ or any of the multi-compartment packaging systems with compartments as described for example in U.S. Pat. No. 6,190,421, col 2, I. 16 to 31.

The pH value of the ready-to-use dyeing compositions is usually from 2 to 11, preferably from 5 to 10.

Preferably dyeing compositions, which are not stable to reduction, are prepared with oxidizing agent free compositions just before the dyeing process.

One preferred embodiment of the present invention relates to the formulation of dyes, wherein the functionalized particles of the invention are in powder form.

Powder formulations are preferably used if stability and/or solubility problems arise, as for example described in DE 197 13 698, p. 2, I. 26 to 54 and p. 3, I. 51 to p. 4, I. 25, and p. 4, I. 41 to p. 5 I. 59.

Suitable cosmetic hair-care formulations are hair-treatment preparations, e.g. hair-washing preparations in the form of shampoos and conditioners, hair-care preparations, e.g. pre-treatment preparations or leave-on products such as sprays, creams, gels, lotions, mousses and oils, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colorants, preparations containing self-oxidizing dyes, or natural hair colorants, such as henna or camomile.

For use on human hair, the dyeing compositions of the present invention can usually be incorporated into an aqueous cosmetic carrier. Suitable aqueous cosmetic carriers include, for example W/O, O/W, O/W/O, W/O/W or PIT emulsions and all kinds of microemulsions, creams, sprays, emulsions, gels, powders and also surfactant-containing foaming solutions, e.g. shampoos or other preparations, that are suitable for use on keratin-containing fibers. Such forms of use are described in detail in Research Disclosure 42448 (August 1999). If necessary, it is also possible to incorporate the dyeing compositions into anhydrous carriers, as described, for example, in U.S. Pat. No. 3,369,970, especially col 1, I. 70 to col 3, I. 55. The dyeing compositions according to the invention are also excellently suitable for the dyeing method described in DE-A-3 829 870 using a dyeing comb or a dyeing brush.

The constituents of the aqueous carrier are present in the dyeing compositions of the present invention in the customary amounts, for example emulsifiers may be present in the dyeing compositions in concentrations from 0.5 to 30% b.w. and thickeners in concentrations from 0.1 to 25% b.w. of the total dyeing composition.

Further carriers for dyeing compositions are for example described in “Dermatology”, edited by Ch. Culnan, H. Maibach, Verlag Marcel Dekker Inc., New York, Basle, 1986, Vol. 7, Ch. Zviak, The Science of Hair Care, chapter 7, p. 248-250, especially on p. 243, I. 1 to p. 244, I. 12.

A shampoo has, for example, the following composition:

0.01 to 5% b.w. of a functionalized particle of the invention;
8% b.w of disodium PEG-5 laurylcitrate Sulfosuccinate, Sodium Laureth Sulfate;
20% b.w. of sodium cocoamphoacetate;
0.5% b.w. of methoxy PEG/PPG-7/3 aminopropyl dimethicone;
0.3% b.w. of hydroxypropyl guar hydroxypropytrimonium chloride;
2.5% b.w. of PEG-200 hydrogenated glyceryl palmate; PEG-7 glyceryl cocoate;
0.5% b.w. of PEG-150 distearate;
2.2. % b.w of citric acid;
perfume, preservatives; and
water ad 100%.

The functionalized particles of the invention may be stored in a liquid to paste-like preparation (aqueous or non-aqueous) or in the form of a dry powder.

When the functionalized particles of the invention and adjuvants are stored together in a liquid preparation, the preparation should be substantially anhydrous in order to reduce reaction of the compounds.

The dyeing compositions according to the invention may comprise any active ingredients, additives or adjuvants known for such preparations, like surfactants, solvents, bases, acids, perfumes, polymeric adjuvants, thickeners and light stabilisers.

The following adjuvants are preferably used in the hair dyeing compositions of the present invention:
non-ionic polymers, for example vinylpyrrolidone/vinyl acrylate copolymers, polyvinyl-pyrrolidone and vinylpyrrolidone/vinyl acetate copolymers and polysiloxanes;
cationic polymers, such as quaternised cellulose ethers, polysiloxanes having quaternary groups, dimethyldiallylammonium chloride polymers, copolymers of dimethyldiallyl-ammonium chloride and acrylic acid, as available commercially under the name Merquat® 280 and the use thereof in hair dyeing as described, for example, in DE-A-4 421 031, especially p. 2, I. 20 to 49, or EP-A-953 334;
acrylamide/dimethyldiallylammonium chloride copolymers, diethyl-sulfate-quaternised dimethylaminoethyl methacrylate/vinylpyrrolidone copolymers, vinylpyrrolidone/-imidazolinium methochloride copolymers;
quaternised polyvinyl alcohol;
zwitterionic and amphoteric polymers, such as acrylamido-propyltrimethylammonium chloride/acrylate copolymers and octylacrylamide/methyl methacrylate/tert-butylaminoethyl methacrylate/2-hydroxypropyl methacrylate copolymers;
anionic polymers, such as, for example, polyacrylic acids, crosslinked polyacrylic acids, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert-butyl acrylamide terpolymers;
thickeners, such as agar, guar gum, alginates, xanthan gum, gum arabic, karaya gum, locust bean flour, linseed gums, dextrans, cellulose derivatives, e.g. methyl cellulose, hydroxyalkyl cellulose and carboxymethyl cellulose, starch fractions and derivatives, such amylose, amylopectin and dextrins, clays, e.g. bentonite or fully synthetic hydrocolloids such as, for example, polyvinyl alcohol;
structuring agents, such as glucose and maleic acid;
hair-conditioning compounds, such as phospholipids, for example soya lecithin, egg lecithin, cephalins, silicone oils, and conditioning compounds, such as those described in DE-A-19 729 080, especially p. 2, I. 20 to 49, EP-A-834 303, especially p. 2, I. 18-p. 3, I. 2, or EP-A-312 343, especially p. 2, I. 59-p. 3, I. 11;
protein hydrolysates, especially elastin, collagen, keratin, milk protein, soya protein and wheat protein hydrolysates, condensation products thereof with fatty acids and also quaternised protein hydrolysates;
perfume oils, dimethyl isosorbitol and cyclodextrins,
solubilisers, such as ethanol, isopropanol, ethylene glycol, propylene glycol, glycerol and diethylene glycol,
anti-dandruff active ingredients, such as piroctones, olamines and zinc Omadine,
substances for adjusting the pH value;
panthenol, pantothenic acid, allantoin, pyrrolidonecarboxylic acids and salts thereof, plant extracts and vitamins;
cholesterol;
light stabilisers and UV absorbers as listed in Table below:

TABLE 1
UV absorbers which may be use in the dyeing compositions of the present invention

| No. | Chemical Name | CAS No. |
|---|---|---|
| 1 | (+/−)-1,7,7-trimethyl-3-[(4-methylphenyl)methylene]bicyclo- | 36861-47-9 |
| | [2.2.1]heptan-2-one | |
| 2 | 1,7,7-trimethyl-3-(phenylmethylene)bicyclo[2.2.1]heptan-2-one | 15087-24-8 |
| 3 | (2-Hydroxy-4-methoxyphenyl)(4-methylphenyl)methanone | 1641-17-4 |
| 4 | 2,4-dihydroxybenzophenone | 131-56-6 |
| 5 | 2,2′,4,4′-tetrahydroxybenzophenone | 131-55-5 |
| 6 | 2-Hydroxy-4-methoxy benzophenone; | 131-57-7 |
| 7 | 2,2′-dihydroxy-4,4′-dimethoxybenzophenone | 131-54-4 |
| 8 | 2,2′-Dihydroxy-4-methoxybenzophenone | 131-53-3 |
| 9 | 1-[4-(1,1-dimethylethyl)phenyl]-3-(4-methoxyphenyl)propane-1,3- | 70356-09-1 |
| | dione | |
| 10 | 3,3,5-Trimethyl cyclohexyl-2-hydroxy benzoate | 118-56-9 |
| 11 | Isopentyl p-methoxycinnamate | 71617-10-2 |
| 12 | Menthyl-o-aminobenzoate | 134-09-8 |
| 13 | Menthyl salicylate | 89-46-3 |
| 14 | 2-Ethylhexyl 2-cyano,3,3-diphenylacrylate | 6197-30-4 |
| 15 | 2-ethylhexyl 4-(dimethylamino)benzoate | 21245-02-3 |
| 16 | 2-ethylhexyl 4-methoxycinnamate | 5466-77-3 |
| 17 | 2-ethylhexyl salicylate | 118-60-5 |
| 18 | Benzoic acid, 4,4′,4″-(1,3,5-triazine-2,4,6-triyltriimino)tris-,tris(2- | 88122-99-0 |
| | ethylhexyl)ester; 2,4,6-Trianilino-(p-carbo-2′-ethylhexyl-1′-oxi)- | |
| | 1,3,5-triazine | |
| 19 | Benzoic acid, 4-amino-, ethyl ester, polymer with oxirane | 113010-52-9 |
| 20 | 2-Propenamide, N-[[4-[(4,7,7-trimethyl-3-oxobicyclo[2.2.1]hept-2- | 147897-12-9 |
| | ylidene)methyl]phenyl]methyl]-, homopolymer | |
| 21 | Triethanolamine salicylate | 2174-16-5 |
| 22 | 2,2′-Methylene-bis-[6-(2H-benzotriazol-2-yl)-4-(1,1,3,3-tetramethyl- | 103597-45-1 |
| | butyl)-phenol] | |
| 23 | 2,4-bis{[4-(2-ethylhexyloxy)-2-hydroxy]-phenyl}-6-(4- | 187393-00-6 |
| | methoxyphenyl)-(1,3,5)-triazine (Tinosorb S) | |
| 24 | Benzoic acid, 4,4′-[[6-[[4-[[(1,1-dimethylethyl)amino]carbonyl]- | 154702-15-5 |
| | phenyl]amino]1,3,5-triazine-2,4-diyl]diimino]bis-, bis(2-ethylhexyl)- | |
| | ester | |
| 25 | Phenol, 2-(2H-benzotriazol-2-yl)-4-methyl-6-[2-methyl-3-[1,3,3,3- | 155633-54-8 |
| | tetramethyl-1-[(trimethylsilyl)oxy]disiloxanyl]propyl]- | |
| 26 | Dimethicodiethylbezalmalonate | 207574-74-1 |
| 27 | Benzoic acid, 2-[4-(diethylamino)-2-hydroxybenzoyl]-, hexyl ester | 302776-68-7 |
| 28 | 1,3,5-Triazine, 2,4,6-tris(4-methoxyphenyl)- | 7753-12-0 |
| 29 | 1,3,5-Triazine, 2,4,6-tris[4-[(2-ethylhexyl)oxy]phenyl]- | 208114-14-1 |
| 30 | 2-Propenoic acid, 3-(1H-imidazol-4-yl)- | 104-98-3 |
| 31 | Benzoic acid, 2-hydroxy-, [4-(1-methylethyl)phenyl]methyl ester | 94134-93-7 |
| 32 | 1,2,3-Propanetriol, 1-(4-aminobenzoate) | 136-44-7 |
| 33 | Benzeneacetic acid, 3,4-dimethoxy-a-oxo- | 4732-70-1 |
| 34 | 2-Propenoic acid, 2-cyano-3,3-diphenyl-, ethyl ester | 5232-99-5 |
| 35 | Anthralinic acid, p-menth-3-yl ester | 134-09-8 |
| 36 | 1,3,5-Triazine-2,4,6-triamine, N,N′-bis[4-[5-(1,1-dimethylpropyl)-2- | 288254-16-0 |
| | benzoxazolyl]phenyl]-N″-(2-ethylhexyl)- or Uvasorb K2A | |
| 37 | 2-Hydroxy-4-methoxy benzophenone-5-sulfonic acid | 4065-45-6 |
| 38 | Alpha-(2-oxoborn-3-ylidene)toluene-4-sulphonic acid and its salts | 56039-58-8 |
| 39 | Methyl N,N,N-trimethyl-4-[(4,7,7-trimethyl-3-oxobicyclo[2,2,1]hept- | 52793-97-2 |
| | 2-ylidene)methyl]anilinium sulphate; | |
| 40 | 4-aminobenzoic acid | 150-13-0 |
| 41 | 2-phenyl-1H-benzimidazole-5-sulphonic acid | 27503-81-7 |
| 42 | 3,3′-(1,4-phenylenedimethylene)bis[7,7-dimethyl-2-oxo- | 90457-82-2 |
| | bicyclo[2.2.1]heptane-1-methanesulfonic acid] | |
| 43 | 1H-Benzimidazole-4,6-disulfonic acid, 2,2′-(1,4-phenylene)bis-, | 180898-37-7 |
| | disodium salt | |
| 44 | Benzenesulfonic acid, 3-(2H-benzotriazol-2-yl)-4-hydroxy-5-(1- | 92484-48-5 |
| | methylpropyl)-, monosodium salt | |
| 45 | 1-Dodecanaminium, N-[3-[[4-(dimethylamino)benzoyl]amino]- | 156679-41-3 |
| | propyl]N,N-dimethyl-, salt with 4-methylbenzenesulfonic acid (1:1) | |
| 46 | 1-Propanaminium, N,N,N-trimethyl-3-[(1-oxo-3-phenyl-2-propenyl)- | 177190-98-6 |
| | amino]-, chloride | |
| 47 | 1H-Benzimidazole-4,6-disulfonic acid, 2,2′-(1,4-phenylene)bis- | 170864-82-1 |
| 48 | 1-Propanaminium, 3-[[3-[3-(2H-benzotriazol-2-yl)-5-(1,1-dimethyl- | 340964-15-0 |
| | ethyl)-4-hydroxyphenyl]-1-oxopropyl]amino]-N,N-diethyl-N-methyl-, | |
| | methyl sulfate (salt) | |
| 49 | 2,2′-bis(1,4-phenylene)-1H-benzimidazole-4,6-disulphonic acid mono | 349580-12-7, |
| | sodium salt or Disodium phenyl dibenzimidazole tetrasulfonate | |
| | or Neoheliopan AP | |

The use of UV absorbers can effectively protect natural and dyed hair from the damaging rays of the sun and increase the wash fastness of dyed hair.

Furthermore, the following UV absorbers or combinations may be used in the dyeing compositions according to the invention:
cationic benzotriazole UV absorbers as for example described in WO 01/36396 especially on p. 1, I. 20 to p. 2, I. 24, and preferred on p. 3 to 5, and on p. 26 to 37;
cationic benzotriazole UV in combination with antioxidants as described in WO 01/36396, especially on p. 11, I. 14 to p. 18;
UV absorbers in combination with antioxidants as described in U.S. Pat. No. 5,922,310, especially in col 2, I. 1 to 3;
UV absorbers in combination with antioxidants as described in U.S. Pat. No. 4,786,493, especially in col 1, 42 to col 2, I. 7, and preferred in col 3, 43 to col 5, I. 20;
combination of UV absorbers as described in U.S. Pat. No. 5,830,441, especially in col 4, I. 53 to 56;
combination of UV absorbers as described in WO 01/36396, especially on p. 11, I. 9 to 13; or
triazine derivatives as described in WO 98/22447, especially on p. 1, I. 23 to p. 2, I. 4, and preferred on p. 2, I. 11 to p. 3, I. 15 and most preferred on p. 6 to 7, and 12 to 16.

Suitable cosmetic preparations may usually contain 0.05 to 40% b.w., preferably from 0.1 to 20% b.w., based on the total weight of the composition, of one or more UV absorbers.

Further ingredients can be:
consistency regulators, such as sugar esters, polyol esters or polyol alkyl ethers;
fats and waxes, such as spermaceti, beeswax, montan wax, paraffins, fatty alcohols and fatty acid esters;
fatty alkanolamides;
polyethylene glycols and polypropylene glycols having a molecular weight of from 150 to 50 000, for example such as those described in EP-A-801 942, especially p. 3, I. 44 to 55,
complexing agents, such as EDTA, NTA and phosphonic acids,
swelling and penetration substances, such as polyols and polyol ethers, as listed extensively, for example, in EP-A-962 219, especially p. 27, I. 18 to 38, for example glycerol, propylene glycol, propylene glycol monoethyl ether, butyl glycol, benzyl alcohol, carbonates, hydrogen carbonates, guanidines, ureas and also primary, secondary and tertiary phosphates, imidazoles, tannins, pyrrole;
opacifiers, such as latex;
pearlising agents, such as ethylene glycol mono- and di-stearate;
propellants, such as propane-butane mixtures, N₂O, dimethyl ether, CO₂ and air;
antioxidants; preferably the phenolic antioxidants and hindered nitroxyl compounds disclosed in ip.com (IPCOM # 000033153D);
sugar-containing polymers, as described in EP-A-970 687;
quaternary ammonium salts, as described in WO 00/10517;
Bacteria inhibiting agents, like preservatives that have a specific action against gram-positive bacteria, such as 2,4,4′-trichloro-2′-hydroxydiphenyl ether, chlorhexidine (1,6-di(4-chlorophenyl-biguanido)hexane) or TCC (3,4,4′-trichlorocarbanilide). A large number of aromatic substances and ethereal oils also have antimicrobial properties. Typical examples are the active ingredients eugenol, menthol and thymol in clove oil, mint oil and thyme oil. A natural deodorising agent of interest is the terpene alcohol farnesol (3,7,11-trimethyl-2,6,10-dodecatrien-1-ol), which is present in lime blossom oil. Glycerol monolaurate has also proved to be a bacteriostatic agent. The amount of the additional bacteria-inhibiting agents present is usually from 0.1 to 2% b.w., based on the solids content of the preparations.

The dyeing compositions according to the present invention generally comprise at least one surfactant.

Suitable surfactants are zwitterionic or ampholytic, or more preferably anionic, non-ionic and/or cationic surfactants.

Suitable anionic surfactants in the dyeing compositions according to the present invention include all anionic surface-active substances that are suitable for use on the human body. Such substances are characterised by an anionic group that imparts water solubility, for example a carboxylate, sulfate, sulfonate or phosphate group, and a lipophilic alkyl group having approximately 10 to 22 carbon atoms. In addition, glycol or polyglycol ether groups, ester, ether and amide groups and also hydroxy groups may be present in the molecule. The following are examples of suitable anionic surfactants, each in the form of sodium, potassium or ammonium salts or mono-, di- or tri-alkanolammonium salts having 2 or 3 carbon atoms in the alkanol group:
linear fatty acids having 10 to 22 carbon atoms (soaps),
ether carboxylic acids of formula R—O—(CH₂—CH₂—O)ₓ—CH₂—COOH, in which R is a l.ar alkyl group having 10 to 22 carbon atoms and x=0 or from 1 to 16,
acyl sarcosides having 10 to 18 carbon atoms in the acyl group,
acyl taurides having 10 to 18 carbon atoms in the acyl group,
acyl isothionates having 10 to 18 carbon atoms in the acyl group,
sulfosuccinic mono- and di-alkyl esters having 8 to 18 carbon atoms in the alkyl group and sulfosuccinic monoalkylpolyoxyethyl esters having 8 to 18 carbon atoms in the alkyl group and from 1 to 6 oxyethyl groups,
linear alkane sulfonates having 12 to 18 carbon atoms,
linear α-olefin sulfonates having 12 to 18 carbon atoms,
α-sulfo fatty acid methyl esters of fatty acids having 12 to 18 carbon atoms,
alkyl sulfates and alkyl polyglycol ether sulfates of formula R′—O(CH₂—CH₂—O)_{x′}—SO₃H, in which R′ is a preferably l.ar alkyl group having 10 to 18 carbon atoms and x′=0 or from 1 to 12,
mixtures of surface-active hydroxysulfonates according to DE-A-3 725 030;
sulfated hydroxyalkylpolyethylene and/or hydroxyalkylenepropylene glycol ethers according to DE-A-3 723 354, especially p. 4, I. 42 to 62,
sulfonates of unsaturated fatty acids having 12 to 24 carbon atoms and 1 to 6 double bonds according to DE-A-3 926 344, especially p. 2, I. 36 to 54,
esters of tartaric acid and citric acid with alcohols which are addition products of approximately from 2 to 15 molecules of ethylene oxide and/or propylene oxide with fatty alcohols having from 8 to 22 carbon atoms, or
anionic surfactants, as described in WO 00/10518, especially p. 45, I. 11 to p. 48, I. 3.

Preferred anionic surfactants are alkyl sulfates, alkyl polyglycol ether sulfates and ether carboxylic acids having 10 to 18 carbon atoms in the alkyl group and up to 12 glycol ether groups in the molecule, and also especially salts of saturated and especially unsaturated C₈-C₂₂-carboxylic acids, such as oleic acid, stearic acid, isostearic acid and palmitic acid.

Surface-active compounds that carry at least one quaternary ammonium group and at least one —COO⁻ or —SO₃⁻ group in the molecule are terminated zwitterionic surfactants. Preference is given the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazol having from 8 to 18 carbon atoms in the alkyl or acyl group and also cocoacylaminoethylhydroxyethylcarboxymethyl glycinate. A preferred zwitterionic surfactant is the fatty acid amide derivative known by the CTFA name cocoamidopropyl betaine.

Ampholytic surfactants are surface-active compounds that, in addition to a C₈-C₁₈-alkyl or acyl group, contain at least one free amino group and at least one —COOH or —SO₃H group in the molecule and are capable of forming internal salts. Examples of suitable ampholytic surfactants include N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids, each having approximately from 8 to 18 carbon atoms in the alkyl group. Ampholytic surfactants to which special preference is given are N-cocoalkylaminopropionate, cocoacylaminoethylaminopropionate and C₁₂-C₁₈acylsarcosine. Suitable non-ionic surfactants are described in WO 00/10519, especially p. 45, I. 11 to p. 50, I. 12. Non-ionic surfactants contain as hydrophilic group, for example, a polyol group, a polyalkylene glycol ether group or a combination of polyol and polyglycol ether groups. Such compounds are, for example:
addition products of 2 to 30 mol of ethylene oxide and/or 0 to 5 mol of propylene oxide with l.ar fatty alcohols having 8 to 22 carbon atoms, with fatty acids having 12 to 22 carbon atoms and with alkylphenols having 8 to 15 carbon atoms in the alkyl group,
C₁₂-C₂₂ fatty acid mono- and di-esters of addition products of 1 to 30 mol of ethylene oxide with glycerol,
C₈-C₂₂alkyl-mono- and -oligo-glycosides and ethoxylated analogues thereof,
addition products of 5 to 60 mol of ethylene oxide with castor oil and hydrogenated castor oil,
addition products of ethylene oxide with sorbitan fatty acid esters,
addition products of ethylene oxide with fatty acid alkanolamides.

The surfactants which are addition products of ethylene and/or propylene oxide with fatty alcohols or derivatives of such addition products may either be products having a “normal” homologue distribution or products having a restricted homologue distribution. “Normal” homologue distribution are mixtures of homologues obtained in the reaction of fatty alcohol and alkylene oxide using alkali metals, alkali metal hydroxides or alkali metal alcoholates as catalysts. Restricted homologue distributions, on the other hand, are obtained when, for example, hydrotalcites, alkali metal salts of either carboxylic acids, alkali metal oxides, hydroxides or alcoholates are used as catalysts.

The use of products having restricted homologue distribution may be preferred.

Examples of cationic surfactants that can be used in the dyeing compositions according to the invention are especially quaternary ammonium compounds. Preference is given to ammonium halides, such as alkyltrimethylammonium chlorides, dialkyldimethylammonium chlorides and trialkylmethylammonium chlorides, for example cetyltrimethylammonium chloride, stearyltrimethylammonium chloride, distearyldimethyl-lammonium chloride, lauryldimethylammonium chloride, lauryldimethylbenzylammonium chloride and tricetylmethylammonium chloride. Further cationic surfactants that can be used in accordance with the invention are quaternised protein hydrolysates.

Also suitable are cationic silicone oils, such as, for example, the commercially available products Q2-7224 (manufacturer: Dow Corning; a stabilised trimethylsilylamodimethicone), Dow Corning 929 emulsion (comprising a hydroxylamino-modified silicone, which is also referred to as amodimethicone), SM-2059 (manufacturer: General Electric), SLM-55067 (manufacturer: Wacker) and also Abil-Quat 3270 and 3272 (manufacturer: Th. Goldschmidt; diquaternary polydimethylsiloxanes, quaternium-80), or silicones, as described in WO 00/12057, especially p. 45, I. 9 to p. 55, I. 2.

Alkylamidoamines, especially fatty acid amidoamines, such as the stearylamidopropyl-dimethylamine obtainable under the name Tego Amid® 18 are also preferred as surfactants in the present dyeing compositions. They are distinguished not only by a good conditioning action but also especially by their good biodegradability.

Quaternary ester compounds, so-called “esterquats”, such as the methyl hydroxyalkyl-dialkoyloxyalkylammonium methosulfates marketed under the trademark Stepantex®, are also very readily biodegradable.

An example of a quaternary sugar derivative that can be used as cationic surfactant is the commercial product Glucquat 100, according to CTFA nomenclature a “lauryl methyl gluceth-10 hydroxypropyl dimonium chloride”.

The alkyl-group-containing compounds used as surfactants may be single substances, but the use of natural raw materials of vegetable or animal origin is generally preferred in the preparation of such substances, with the result that the substance mixtures obtained have different alkyl chain lengths according to the particular starting material used.

Usually, the dyeing compositions are applied to the keratin-containing fiber in an amount of from 50 to 100 g.

The functionalized particles of the invention are suitable for all-over dyeing of the hair, that is to say when dyeing the hair on a first occasion, and also for re-dyeing subsequently, or dyeing of locks or parts of the hair.

The functionalized particles of the invention are applied on the hair for example by massage with the hand, a comb, a brush, or a bottle, or a bottle, which is combined with a comb or a nozzle.

In the processes for dyeing according to the invention, whether or not dyeing is to be carried out in the presence of a further dye will depend upon the color shade to be obtained.

Further preferred is a process for dyeing keratin-containing fibers which comprises treating the keratin-containing fiber with at least one functionalized particle of the invention, a base and an oxidizing agent.

The oxidation dyeing process usually involves lightening, that is to say that it involves applying to the keratin-containing fibers, at basic pH, a mixture of bases and aqueous hydrogen peroxide solution, leaving the applied mixture to stand on the hair and then rinsing the hair. It allows, particularly in the case of hair dyeing, the melanin to be lightened and the hair to be dyed.

Lightening the melanin has the advantageous effect of creating a unified dyeing in the case of grey hair, and, in the case of naturally pigmented hair, of bringing out the color, that is to say of making it more visible.

In general, the oxidizing agent containing composition is left on the fiber for 0 to 15 minutes, in particular for 0 to 5 minutes at 15 to 45° C., usually in amounts of 30 to 200 g.

Oxidizing agents are for example persulfate or dilute hydrogen peroxide solutions, hydrogen peroxide emulsions or hydrogen peroxide gels, alkaline earth metal peroxides, organic peroxides, such as urea peroxides, melamine peroxides, or alkalimetalbromat fixations are also applicable if a shading powder on the basis of semi-permanent, direct hair dyes is used.

Further preferred oxidizing agents are
oxidizing agents to achieve lightened coloration, as described in WO 97/20545, especially p. 9, I. 5 to 9,
oxidizing agents in the form of permanent-wave fixing solution, as described in DE-A-19 713 698, especially p. 4, I. 52 to 55, and 1.60 and 61 or EP-A-1062940, especially p. 6, I. 41 to 47 (and in the equivalent WO 99/40895).

Most preferred as oxidizing agent is hydrogen peroxide, preferably used in a concentration from about 2 to 30%, more preferably about 3 to 20% by, and most preferably from 6 to 12% b.w. of the corresponding composition.

The oxidizing agents may be present in the dyeing compositions according to the invention preferably in an amount from 0.01% to 6%, especially from 0.01% to 1%, based on the total dyeing composition.

In general, the dyeing with an oxidative agent is carried out in the presence of a base, for example ammonia, alkali metal carbonates, earth metal (potassium or lithium) carbonates, alkanol amines, such as mono-, di- or triethanolamine, alkali metal (sodium) hydroxides, earth metal hydroxides or compounds of the formula

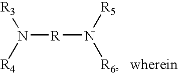

R is a propylene residue, which may be substituted with OH or C₁-C₄alkyl,
R₃, R₄, R₅ and R₆ are independently or dependently from each other hydrogen, C₁-C₄alkyl or hydroxy-(C₁-C₄)alkyl.

The pH-value of the oxidizing agent containing composition is usually about 2 to 7, and in particular about 2 to 5.

One preferred method of applying formulations comprising the functionalized particles of the invention on the keratin-containing fiber, preferably human hair, is by using a multi-compartment dyeing device or “kit” or any other multi-compartment packaging system, as described for example in WO 97/20545 on p. 4, I. 19 to I. 27.

The first compartment contains for example at least one functionalized particle of the invention and optionally further direct dyes and a basifying agent, and in the second compartment an oxidizing agent; or in the first compartment at least one functionalized particle of the invention and optionally further direct dyes, in the second compartment a basifying agent and in the third compartment an oxidizing agent.

Generally the hair is rinsed after treatment with the dyeing solution and/or permanent-wave solution.

A further preferred embodiment of the present invention relates to a method of dyeing hair with oxidative dyes, which comprises
a. mixing at least one functionalized particle of the invention and optionally at least one coupler compound and at least one developer compound, and an oxidizing agent, which optionally contains at least one further dye, and
b. contacting the keratin-containing fibers with the mixture as prepared in step a.

The pH-value of the oxidizing agent free composition is usually from 3 to 11, and in particular from 5 to 10, and most particular about 9 to 10.

Preferably, a ready-to-use composition is prepared according to a first preferred embodiment by a process which comprises a preliminary step which involves separately storing, on the one hand, a composition (A) comprising, in a medium which is suitable for dyeing, at least one developer compound, especially selected from para-phenylenediamines and bis(phenyl)-alkylenediamines, and the acid-addition salts thereof, at least one coupler, especially selected from meta-phenylenediamines and the acid-addition salts thereof, and at least one functionalized particle of the invention, on the other hand, a composition (B) containing, in a medium which is suitable for dyeing, at least one oxidizing agent and mixing (A) and (B) together immediately before applying this mixture to the keratin-containing fibers.

According to a second preferred embodiment for the preparation of the ready-to-use dye composition, the process includes a preliminary step which involves separately storing, on the one hand, a composition (A) comprising, in a medium which is suitable for dyeing, at least one developer compound, especially selected from para-phenylenediamines and bis(phenyl)alkylenediamines, and the acid-addition salts thereof, at least one coupler compound, especially selected from meta-phenylenediamines and the acid-addition salts thereof; on the other hand, a composition (A′) comprising, in a medium which is suitable for dyeing, at least one functionalized particle of the invention, and, finally, a composition (B) containing, in a medium which is suitable for dyeing, at least one oxidizing agent as defined above, and mixing them together at the time of use immediately before applying this mixture to the keratin-containing fibers.

The composition (A′) used according to this second embodiment may optionally be in powder form, the functionalized particle(s) of the invention (themselves) constituting, in this case, all of the composition (A′) or optionally being dispersed in an organic and/or inorganic pulverulent excipient.

When present in the composition A′, the organic excipient may be of synthetic or natural origin and is selected in particular from crosslinked and non-crosslinked synthetic polymers, polysaccharides such as celluloses and modified or unmodified starches, as well as natural products such as sawdust and plant gums (guar gum, carob gum, xanthan gum, etc.).

When present in the composition (A′), the inorganic excipient may contain metal oxides such as titanium oxides, aluminium oxides, kaolin, talc, silicates, mica and silicas.

A very suitable excipient in the dyeing compositions according to the invention is sawdust.

The powdered composition (A′) may also contain binders or coating products in an amount which preferably does not exceed approximately 3% b.w. relative to the total weight of composition (A′). These binders are preferably selected from oils and liquid fatty substances of inorganic, synthetic, animal or plant origin.

Furthermore, the present invention relates to a process for dyeing of keratin-containing fibers with the functionalized particles of the invention and together with autooxidable compounds and optionally further dyes.

Furthermore, the present invention relates to a process for dyeing of keratin-containing fibers with the functionalized particles of the invention and capped diazotised compounds, which comprises,
a. treating the keratin-containing fibers under alkaline conditions with at least one capped diazotised compound and a coupler compound, and optionally a developer compound and optionally an oxidizing agent, and optionally in the presence of a further dye, and optionally with at least one functionalized particle of the invention; and
b. adjusting the pH in the range of 6 to 2 by treatment with an acid, optionally in the presence of a further dye, and optionally at least one functionalized particle of the invention,
   with the proviso that at least in one of the steps a. and b. at least one functionalized particle of the invention is present.

The capped diazotised compound and coupler compound and optionally the oxidizing agent and developer compound can be applied in any desired order successively, or simultaneously.

Preferably, the capped diazotised compound and the coupler compound are applied simultaneously, in a single composition.

“Alkaline conditions” denotes a pH in the range from 8 to 10, preferably 9-10, especially 9.5-10, which is achieved by the addition of bases, for example sodium carbonate, ammonia or sodium hydroxide.

The bases may be added to the hair, to the dye precursors, the capped diazotised compound and/or the water-soluble coupling component, or to the dyeing compositions comprising the dye precursors.

Acids are for example tartaric acid or citric acid, a citric acid gel, a suitable buffer solution with optionally an acid dye.

The ratio of the amount of alkaline dyeing composition applied in the first stage to that of acid dyeing composition applied in the second stage is preferably about from 1:3 to 3:1, especially about 1:1.

The following Examples serve to illustrate the present invention without limiting the present invention thereto. Unless specified otherwise, parts and percentages relate to weight. The amount of dyes specified are relative to the material being coloured.

### EXAMPLES A

### Process of Preparation

### Example A1

### 3-Aminopropyl Silane Modified Silica Nanoparticles

510 g of Ludox TMA (Helm AG, 34% nanosilica dispersion in water) is mixed with 2490 g ethanol (EtOH). 345 g 3-aminopropyl-trimethoxysilane (Fluka purum) is added drop wise to this homogeneous mixture. After the addition, the mixture is heated to 50° C. for 18 hours. The volume of this mixture is then reduced to about 1 liter by evaporating EtOH/H₂O in the rotary evaporator. A total of 4 liter hexane is added, the mixture shaken vigorously and the two phases separated in a separation funnel to remove unreacted aminosilane. The aqueous/ethanolic lower phase is concentrated to a wet paste in the rotary evaporator in vacuo and then re-suspended in 1 liter ethanol. A total of 1199 g solution is obtained with a solid content of 27.3 wt. %.

### Analytics:

Thermogravimetric analysis (TGA; heating rate: 10° C./min from 50° C. to 600° C.): Weight loss: 25.2% corresponding to the organic material.

Elemental analysis: found: C, 17.68%; H, 4.65%; N, 6.73%, corresponding to an organic content of 28.1% in relatively good agreement to the TGA value.

Transmission Electron Microscopy (TEM): An average diameter of 35-40 nm is obtained for the individual nanoparticles.

Dynamic light scattering (DLS): Average diameter d=90-110 nm.

### Example A2

### 3-Aminopropyl Silane Modified Alumina Nanoparticles

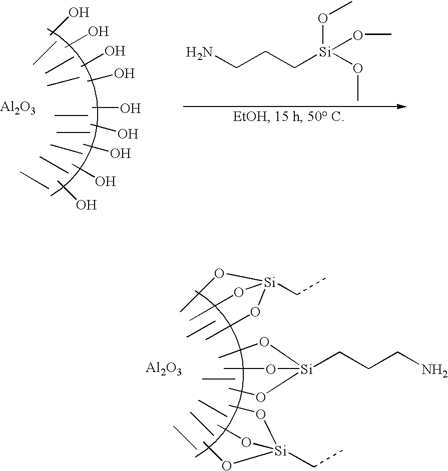

150 g of alumina nanoparticles (Nyacol Corp., Nyacol A120 DW, 22% nanoalumina dispersion in water) is mixed with 250 ml ethanol (EtOH). 27 g 3-aminopropyltrimethoxysilane (Fluka purum) is added drop wise to this homogeneous mixture. After the addition, the mixture is heated to 50° C. for 15 hours. The volume of this mixture is then reduced to about 1 liter by evaporating EtOH/H₂O in the rotary evaporator. The obtained solid is redispersed in EtOH to an 11.4 wt. % opaque dispersion.

### Analytics:

Thermogravimetric analysis (TGA; heating rate: 10° C./min from 50° C. to 800° C.): Weight loss: 27.9 wt. % corresponding to the organic material.

Elemental analysis: found: N, 4.16 wt. %: corresponding to an organic content of 17.3 wt. %.

The difference between TGA and EA results is due to the loss of water out of the inorganic matrix and water generated from condensation processes on the surface during thermal treatment.

Transmission Electron Microscopy (TEM): An average diameter of 50 to 60 nm is obtained for the individual primary nanoparticles.

Dynamic light scattering (DLS): Average diameter d=164 nm.

### Example A3

### a) Preparation of Anthraquinone Dye of Formula (101) [Precursor]

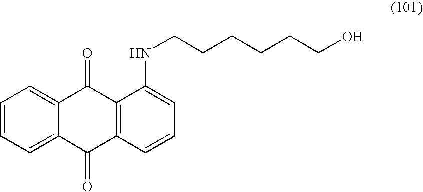

A mixture of 6.0 g of 1-fluoro-anthraquinone, 3.4 g hexanolamine (FLUKA) and 4.0 g potassium carbonate are heated with stirring to 95° C. for 25 hours until the starting fluoride is consumed. The reaction mixture is then filtered. The red residue is taken up in ethyl acetate and extracted successively with 1 N hydrogen chloride (3 times), saturated sodium hydrogen chloride solution and brine. Evaporation of the solvent leaves a red residue which is purified over a short silica gel column (230-400 mesh, FLUKA) and eluent (hexane-ethyl acetate 10:2 (v/v)) to give 6.3 g of the desired red alcohol of formula (101).

¹H-NMR (CDCl₃, 300 MHz): 1.40-1.81 (m, 8H); 3.26 (ddd, 2H); 3.66 (t, 2H); 6.98 (dd, 1H); 7.45 (ddd, 1H); 7.50 (dd, 1H); 7.62-773 (m, 2H); 8.15-8.22 (m, 2H).

¹³C-NMR (CDCl₃, 75 MHz): 25.85; 27.29; 29.34; 32.79; 43.06; 62.70; 112.94; 115.76; 118.11; 126.78; 126.83; 133.05; 133.13; 134.13; 134.74; 135.18; 135.45; 151.78; 184.06; 184.99.

### b) Preparation of Anthraquinone Dye of Formula (102)

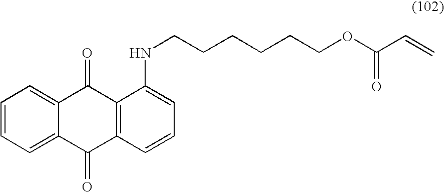

The compound of formula (101) is esterified in the presence of the biocatalyst NOVO 435 (Novozymes, Denmark). At 50° C. and a vacuum at about 450 mbar 10.0 g of the compound of formula (101), 22.2 ml of acrylic acid methyl ester and 5.0 g of the biocatalyst are reacted in 75 ml toluene for 24 hours until all of the starting compound of formula (101) is consumed. The mixture is then filtered, washed with dichloromethane and the solvent evaporated. After vacuum drying 11.5 g of the desired red acrylic ester of formula (102) is obtained.

¹H-NMR (CDCl₃, 300 MHz): 1.35-1.77 (m, 8H); 3.25 (dt, 2H); 4.10 (t, 2H); 5.73 (dd, 1H); 6.04 (dd, 1H); 6.28 (dd, 1H); 6.96 (dd, 1H); 7.44 (dd, 1H); 7.50 (dd, 1H); 7.60 dt, 1H); 7.66 (dt, 1H); 8.14 (m, 2H); 9.64 (broad, t, 1H).

¹³C-NMR (CDCl₃, 75 MHz): 26.15; 27.23; 28.93; 29.40; 43.19; 64.77; 113.11; 115.77; 117.98; 126.83; 126.88; 128.78; 130.67; 133.04; 133.22; 134.06; 134.87; 135.22; 135.43; 151.90; 166.40; 183.87; 185.04.

### Example A4

### a) Preparation of Anthraquinone Dye of Formula (103) [Precursor]

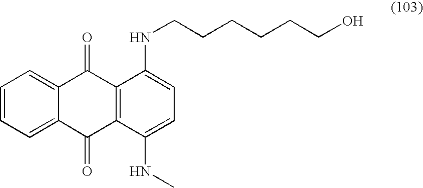

1.0 g of 1-N-methyl, 4-bromo anthraquinone, 1.0 g of 6-aminohexanol (FLUKA), 0.6 g of potassium carbonate and 0.2 g of copper powder are heated to 100° C. in 5 ml of toluene for 26 hours. The reaction mixture is filtered, washed with acetone and the residue dissolved in dichloromethane. The blue solution is applied to a silica gel (230-400 mesh, FLUKA) column and eluted with dichloromethane-methanol 10:2 (v/v) to give 0.5 g of the desired blue alcohol of formula (103).

¹H-NMR (CDCl₃, 300 MHz): 1.32-1.61 (m, 6H); 1.69 (quint., 2H); 2.99 (d, 3H); 3.29 (q, 2H); 3.58 (t, 2H); 7.10 (dd, 2H); 7.60 (dd, 2H); 8.21 (dd, 2H); 10.51 (broad, 1H); 10.64 (broad t, 1H).

¹³C-NMR (CDCl₃, 75 MHz): 25.86; 27.27; 29.83; 29.88; 32.95; 43.11; 63.04; 109.90; 110.09 123.24; 123.69; 126.17 (2×C); 132.10 (2×C); 134.03; 134.68; 146.34; 147.03; 182.35 (2×C).

### b) Preparation of Anthraquinone Dye of Formula (104)

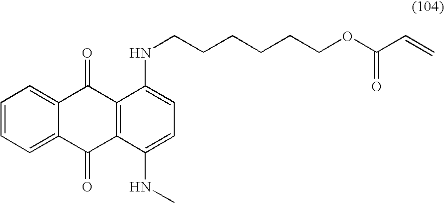

In analogy to Example A3 b), 5.0 g of the alcohol of formula (103) are converted to the ester of formula (104) in the presence of 4.0 g of biocatalyst and methacrylic acid methyl ester. The ester of formula (104) is obtained in 5.8 g after filtration from the catalyst and washing the biocatalyst with dichloromethane.

¹H-NMR (CDCl₃, 300 MHz): 1.35-1.76 (m, 8H); 3.02 (d, 3H); 3.32 (dt, 2H); 4.09 (t, 2H); 5.74 (dd, 1H); 6.04 (dd, 1H); 6.28 (dd, 1H); 7.15 (s, 2H); 7.61 (m, 2H); 8.23 (m, 2H); 10.53 (broad q, 1H); 10.66 (broad t, 1H).

### Example A5

### Anthraquinone Modified Silica Nanoparticles with Cationic Surface Groups

### Reaction Scheme:

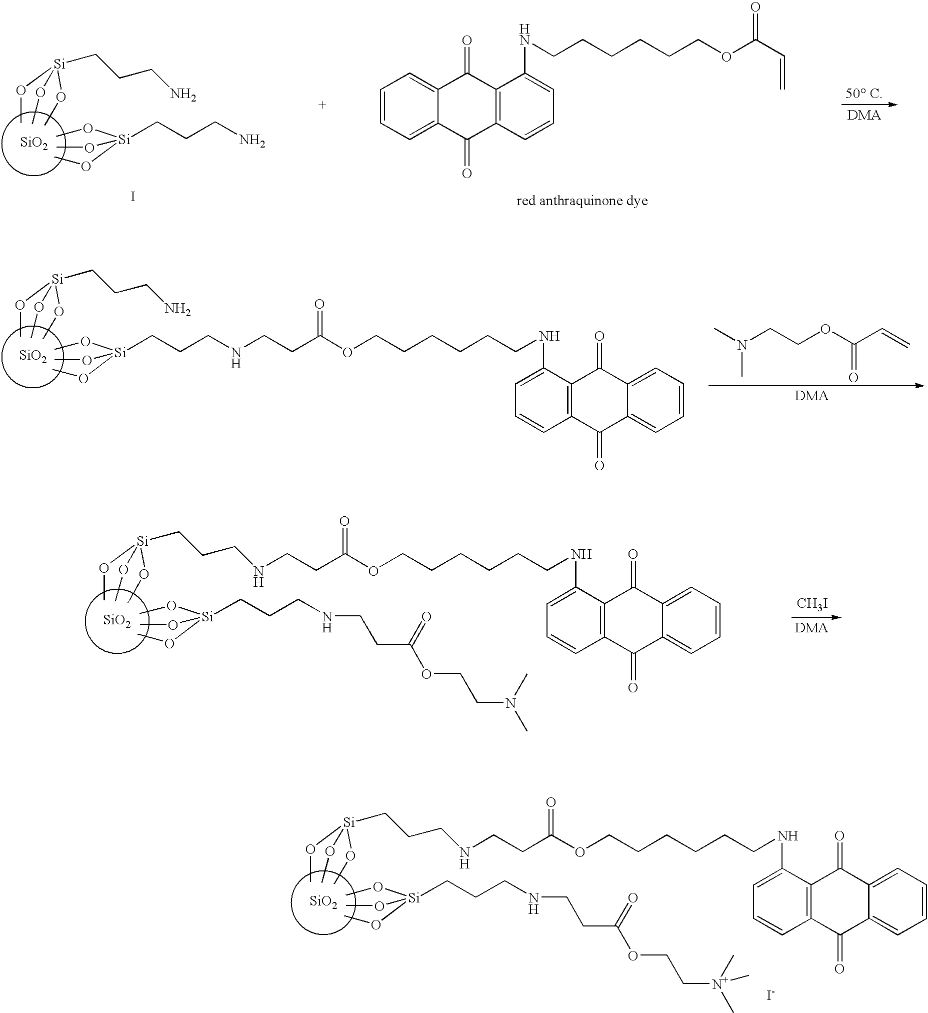

The synthesis is carried out in a round bottom flask. 3.25 g of a dimethyl acetamide (DMA) dispersion containing 26.2 wt. % 3-aminopropyl silane modified silica nanoparticles (obtainable according to Example A1 above, N, 6.73 wt. %) are mixed with a solution of 0.775 g of the anthraquinone compound of formula (102) in 10 g DMA. The reaction mixture is stirred for 15 hours at 50° C. Afterwards, 0.59 g of 2-(dimethylamino)ethyl acrylate are added to the reaction dispersion and again stirred for 15 hours at 50° C. After this time, no acrylic groups can be analyzed by ¹H-NMR. Then, 0.6 g of methyl iodide are added slowly to the dispersion and the reaction is carried out for 15 hours at 50° C. After cooling down, the solvent of the red colored reaction dispersion is evaporated using a rotary evaporator. A dark red resin is obtained which is easily re-dispersible in water.

### Analytics:

Thermogravimetric analysis (TGA; heating rate: 10° C./min from 50° C. to 800° C.): Weight loss: 85.1 wt. % corresponding to the organic material.

Dynamic light scattering (DLS): Average diameter d=65 nm.

UV/VIS: λₘₐₓ=549 nm

### Example A6

### Anthraquinone Modified Silica Nanoparticles with Cationic Surface Groups

### Reaction Scheme:

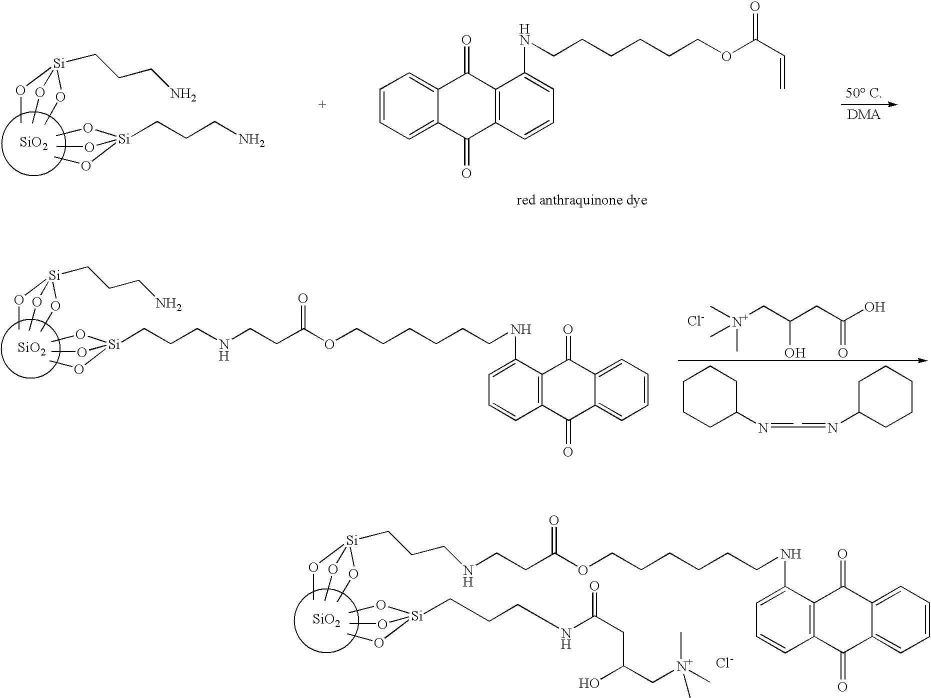

The synthesis is carried out in a round bottom flask. 3.25 g of a DMA dispersion containing 26.2 wt. % 3-aminopropyl silane modified silica nanoparticles (obtainable according to Example A1 above, N, 6.73 wt. %) are mixed with a solution of 0.775 g of the anthraquinone compound of formula (102) in 10 g DMA. The reaction mixture is stirred for 15 hours at 50° C. Afterwards, the reaction mixture is cooled down to 0° C. using an ice/water bath. Then, 0.405 g of the following compound of formula (105)

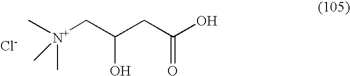

and 0.43 g of 1,3-dicyclohexylcarbodiimide are added to the reaction dispersion and stirred for 5 hours at 0° C. After this time, the reaction dispersion is brought to room temperature. A red dispersion is obtained. After evaporating the solvent with a rotary evaporator the obtained resin is redispersed in 50 ml of a 1:1 water/acetone mixture and centrifuged at 2000 rpm for 20 minutes. The solvent is separated from the solid and fresh solvent is added. The cleaning procedure by using centrifugation is repeated 5 times. Afterwards, the red product is re-dispersed in water to get a dispersion with a solid content of 5 wt. %.

### Analytics:

Thermogravimetric analysis (TGA; heating rate: 10° C./min from 50° C. to 800° C.): Weight loss: 60 wt. % corresponding to the organic material.

Dynamic light scattering (DLS): Average diameter d=127 nm.

UV/VIS: λₘₐₓ=547 nm

### Example A7

### Anthraquinone Modified Silica Nanoparticles with Cationic Surface Groups

1.12 g of a dispersion containing 26.2 wt. % 3-aminopropylsilane modified silica nanoparticles (obtainable according to Example A1, N, 6.73 wt. %) are mixed with 1.9 g of MPEG(8)acrylate (=poly(ethyleneglycol)methyl ether acrylate, CAS 32171-39-4, Aldrich, MW=454) and a solution of 0.3 g of anthraquinone dye acrylate of formula (104) (obtainable according to example A4) in 30 ml ethanol. The reaction mixture is stirred for 15 hours at 50° C. Afterwards, the reaction mixture is cooled down to room temperature. After this time, small signal relating to acrylic groups can be analyzed by ¹H-NMR. The dispersion is washed using centrifugation at 3000 rpm for 15 minutes. The blue solid is washed with ethanol, redispersed in water/ethanol and again centrifugated. This washing procedure is repeated until no free acrylic bonds can be observed in ¹H-NMR analysis. The product is then redispersed in water and 1 ml of 2 mol/l HCl solution is added. The pH of the final blue dispersion is 2.

### Analytics:

Thermographimetric analysis (TGA; heating rate: 10° C./min from 50° C. to 800° C.): Weight loss: 59 wt. % corresponding to the organic material.

Dynamic light scattering (DLS): Average diameter d=225 nm.

### Example A8

### Anthraquinone Modified Silica Nanoparticles with Cationic Surface Groups

3.25 g of a dispersion containing 26.2 wt. % 3-aminopropylsilane modified silica nanoparticles (obtainable according to Example A1, N, 6.73 wt. %) are mixed with 0.93 g of MPEG(8)acrylate (=poly(ethyleneglycol)methyl ether acrylate, CAS 32171-39-4, Aldrich, MW=454) and a solution of 0.78 g of the antraquinone dye acrylate of formula (102) (obtainable according to Example A3) in 40 mL ethanol. The reaction mixture is stirred for 15 hours at 50° C. Afterwards, the reaction mixture is cooled down to room temperature. After this time, small signal relating to acrylic groups can be analyzed by ¹H-NMR. The dispersion is washed using centrifugation at 3000 rpm for 15 minutes. The red solid is washed with ethanol, redispersed in water/ethanol and again centrifuged. This washing procedure is repeated until no free acrylic bonds can be observed in ¹H-NMR analysis. The product is then redispersed in water and 2 mol/l HCl solution is added to adjust the pH value to 2.

### Analytics:

Thermographimetric analysis (TGA; heating rate: 10° C./min from 50° C. to 800° C.): Weight loss: 62 wt. % corresponding to the organic material.

Dynamic light scattering (DLS): Average diameter d=180 nm.

UV/VIS (water): λₘₐₓ=518 nm (pH 2), 524 nm (pH 5).

### Example A9

### Silica Nanoparticles Surface Modified with Cationic Surface Groups and Fe²⁺ Complex Groups

### Reaction Scheme:

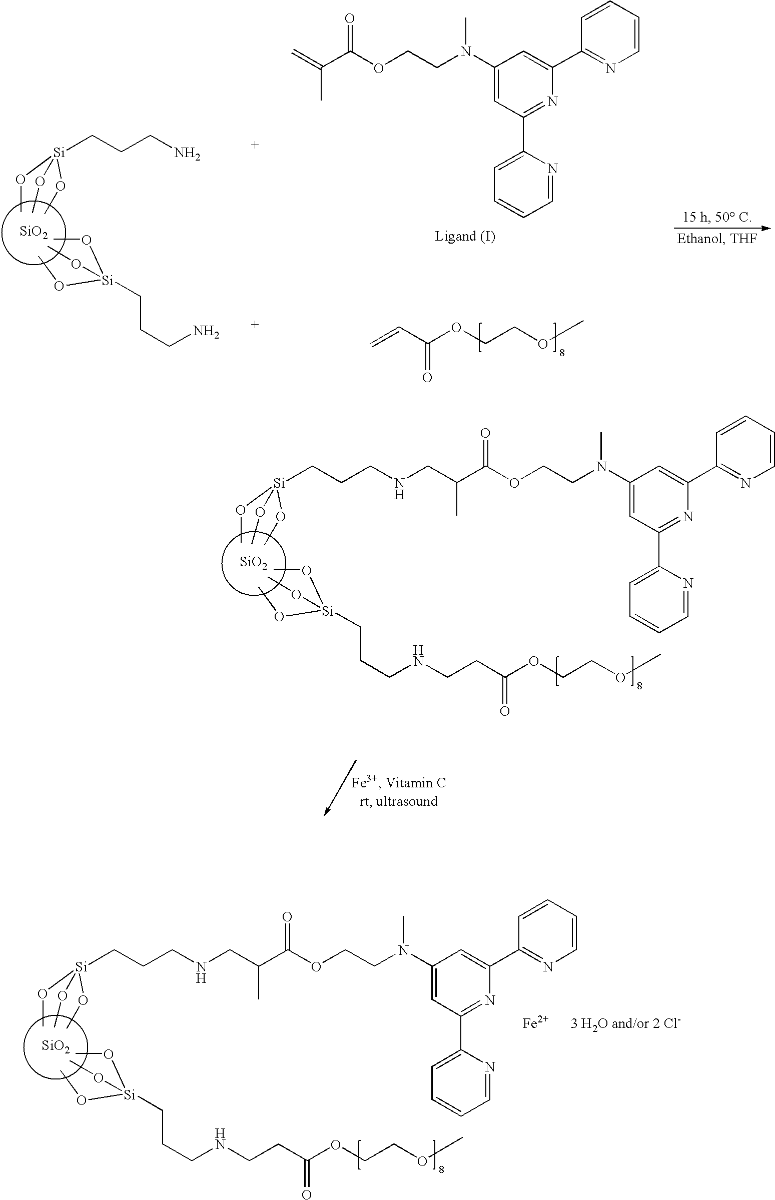

The synthesis described below is carried out in a round bottom flask. 3 g of a dispersion containing 26.2 wt. % 3-aminopropylsilane modified silica nanoparticles (obtainable according to Example A1, N, 6.73 wt. %) are mixed with 1.2 g of MPEG(8)acrylate (=poly(ethyleneglycol)methyl ether acrylate, CAS 32171-39-4, Aldrich, MW=454) and a solution of 0.425 g of ligand (I) [as to the structure of this ligand see the above reaction scheme] in 30 g of THF. The reaction is stirred for 15 hours at 50° C. Afterwards, the reaction mixture is cooled down to room temperature. After this time, no acrylic groups can be analyzed by ¹H-NMR. After evaporating the solvent with a rotary evaporator the obtained resin is redispersed in a mixture of 85 g of water and 3 g of ethanol.

In parallel a fresh 60 mmol solution A of Fe³⁺ is prepared using FeCl₃.6H₂O. In addition, a fresh 60 mmol solution B of vitamin C is prepared.

After adding each 18 ml of solution A and B to the nanoparticle dispersion the colour changes immediately from milky white to violet. Using ultrasonic treatment, enforces the reaction and the complex formation and leads to a dark violet coloured nanoparticle dispersion.

### Analytics:

Thermographimetric analysis (TGA; heating rate: 10° C./min from 50° C. to 800° C.): Weight loss: 79 wt. % corresponding to the organic material.

Dynamic light scattering (DLS): Average diameter d=91 nm.

UV/VIS (water): λₘₐₓ=571 nm.

### Example A10

### Azo Dye Modified Silica Nanoparticles

### Reaction Scheme:

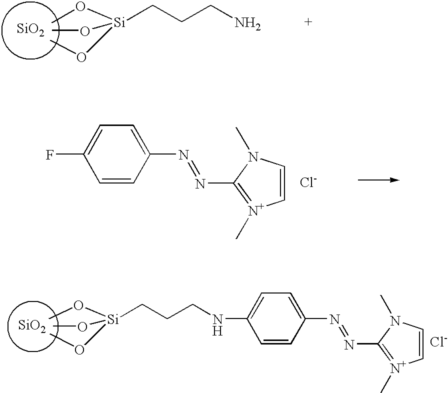

The ethanolic suspension obtained according to Example A1 is concentrated to dryness in vacuo and re-suspended in isopropanol to obtain a suspension with a solid content of 27.8 wt. %. In a round bottom flask equipped with a reflux condenser and a dropping funnel, 5 g of this suspension is heated to reflux temperature. Then a solution of 1.0 g of the compound of formula (106) [prepared as described in WO 2004/076564]

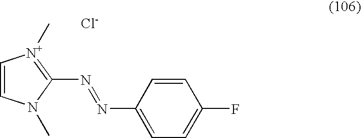

in 40 ml of isopropanol is added over a period of 6 hours at reflux temperature. The resulting red precipitate is filtered off and refluxed for 2 hours with 50 ml of isopropanol. After filtration the product is dried in vacuo to obtain 2.7 g of a red powder, which can be redispersed in water.

### Analytics:

Elemental analysis: found: C, 33.22 wt. %; H, 4.57 wt. %; N, 12.75 wt. %, corresponding to an organic content of 50.54 wt. %.

Dynamic light scattering (DLS): Average diameter d=69 nm.

Scanning Electron Microscope (SEM): The size of the observed particles was in the range of 30 to 70 nm.

UV/VIS (water): λₘₐₓ 505 nm.

If in Example A10 the fluoro-substituted dye of formula (106) is replaced by the methoxy-substituted dye of formula (107) (prepared as in US2004187231)

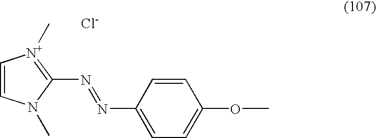

under the same reaction conditions an identical product can be obtained.

If in Examples A5 to A10 3-aminopropyl silane modified alumina nanoparticles (obtainable according to Example A2) are used instead of 3-aminopropyl silane modified silica nanoparticles, corresponding functionalized alumina nanoparticles can be obtained.

### Example A11

A mixture of tetraethoxysilane (18 g), ammonia (25% solution in water, 8.9 g) and ethanol (210 g) is stirred overnight at room temperature. (3-Aminopropyl)trimethoxysilane (5.20 g) is then added to 110 g of the initial dispersion and the mixture is heated to 55° C. overnight. The mixture is then cooled to room temperature and ¾ of the solvents are evaporated. An equal volume of hexane is added, and the mixture centrifuged. The resulting mother liquor is decanted off and the white solid washed again with hexane. These functionalised silica particles are then re-dispersed in ethanol. A mixture of this dispersion (10 ml, 0.17 g solid material) and the compound of formula (106) (0.2 g) and isopropanol (10 ml) is heated to reflux overnight. This mixture is then cooled to room temperature and the red precipitate filtered and washed with ethanol followed by acetone to give the cationic dye functionalised nanoparticles as a red powder (0.155 g). DLS and SEM show monodisperse particles of average diameter 90 nm and about 70 nm respectively. TGA shows loss of 44.065%, corresponding to the organic material and elemental analysis gives 27.71% (C, H, N).

UV/VIS (water): λₘₐₓ 505 nm.

### Examples A12 to A16

Variation of the silane/ammonia/ethanol ratios in the above method of Example A11 gives different silica nanoparticle sizes. Reaction of these differently sized particles in the same way as above in Example A11 gives the cationic dye functionalised nanoparticles of the following approximate diameters:

| Example | Si(OEt)₄ | NH₃ | Ethanol | DLS | TGA | EA |
|---|---|---|---|---|---|---|
| A12 | 6.22 g | 2.5 g | 100 ml | 53 nm | 45.56 | 36.26 |
| A13 | 12.63 g | 11.38 g | 250 ml | 170 nm | 14.469 | 10.41 |
| A14 | 18 g | 8.9 g | 266 ml* | 190 nm | 28.93 | 22.94 |
| A15 | 37.89 g | 11.38 g** | 250 ml | 300 nm | 10.426 | 7.29 |
| A16 | 9.34 g | 19.11 g | 100 ml | 350 nm | 9.408 | 6.23 |

*technical grade ethanol used
**18.75 ml H₂O also added
EA = elemental analysis of the total of C, H and N in weight-% based on the weight of the product.

### Examples A17 to A18

Variation of the amount of the dye of formula (106) used in Example A10 gives particles substituted with a mixture of aminopropyl groups and cationic dyes.

### Reaction Scheme:

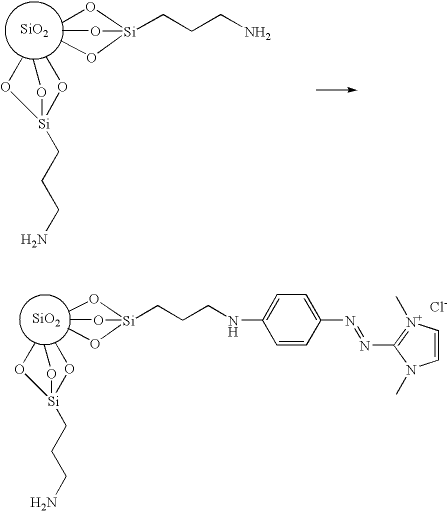

A mixture of aminopropyl-substituted nanoparticles obtainable according to Example A1, the dye of formula (106) and isopropanol is heated to reflux overnight. The mixture is then cooled to room temperature, filtered and washed with isopropanol. This solid is heated to reflux in isopropanol for one more hour, then cooled to room temperature, filtered and washed with isopropanol followed by diethyl ether. This gives the product as a dark red solid.

| | Amount | | | | |
|---|---|---|---|---|---|
| | of dispersion | | | | |
| | according | Amount of the | | | |
| | to Example | dye of formula | | | |
| Example | A1 | (106) | DLS | TGA | EA |
| A17 | 4.75 g | 0.802 g | 51 nm | 46.96 | 38.17 |
| A18 | 4.79 g | 0.482 g | 52 nm | 22.154 | 20.50 |

### Example A19

### Reaction Scheme

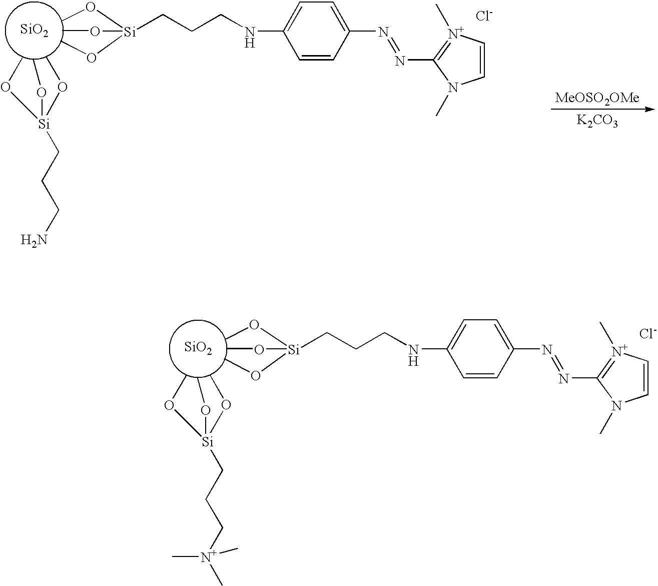

Cationic dye and amino-substituted nanoparticles obtainable according to Example A10 (250 mg), dimethyl sulfate (1.715 g), potassium carbonate (2.257 g) and methanol (100 ml) is heated to reflux overnight. The mixture is then cooled to room temperature and the white precipitate filtered off. Diethyl ether (200 ml) is then added and again the white precipitate is filtered off. Upon standing for 1 hour, the filtrate forms a dark red precipitate, which is filtered off and washed with diethyl ether to give the product as a dark red solid (170 mg).

### Examples A20 and A21

Under the same reaction conditions of Example A19, the products obtainable according to Examples A17 and A18 are treated with dimethyl sulfate (DMS)

| | Starting material of | | |
|---|---|---|---|
| Example | Example | TGA | EA (C, H, N total) |
| A20 | A18 | 36.89 | 19.27 |
| A21 | A17 | 48.48 | 29.97 |

### Example A22

### Reaction Scheme

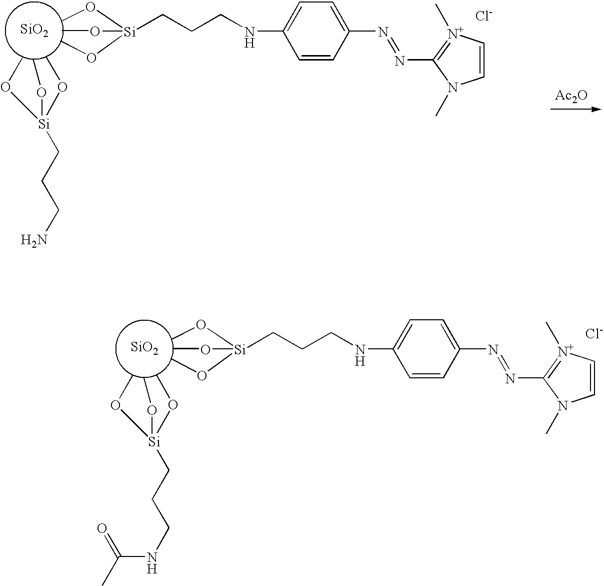

A mixture of the product obtainable according to Example A10 (300 mg) and acetic anhydride (10 ml) is heated to 110° C. for 2 hours. The mixture is then cooled to room temperature, acetone (30 ml) is added and the formed precipitate is filtered and washed with more acetone followed by diethyl ether to give the product as a red solid (240 mg). Elemental analysis shows C, 27.68; H, 4.31; N, 10.54%.

### Example A23

### Step 1: Preparation of the Compounds of Formulae (108) to (110)

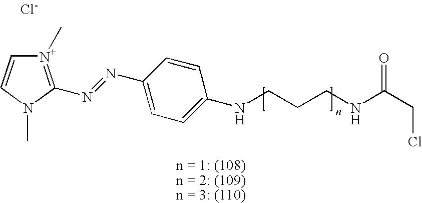

A mixture of the dye of formula (107) (2.0 g), 1,3-diaminopropane (2.22 g) and acetonitrile (15 ml) is heated to 55° C. for 3 hours. The mixture is then cooled to room temperature, 50 ml diethyl ether are added and the red precipitate is filtered and washed with more diethyl ether to give the aminopropyl substituted dye. This aminopropyl-substituted dye (500 mg) is then treated with chloroacetyl chloride (220 mg) and triethylamine (327 mg) in acetonitrile (10 ml) and stirred overnight at room temperature. Evaporation of the solvent followed by column chromatography (SiO₂, eluent H₂O/EtOAc/n-BuOH/HCO₂H) gives the chloroacetamide of formula (108) as a red solid (320 mg).

The compounds of formulae (109) and (110), with C₆ and C₁₂ chains respectively, are prepared in a similar way.

### Step 2: Preparation of the Compound of Formula

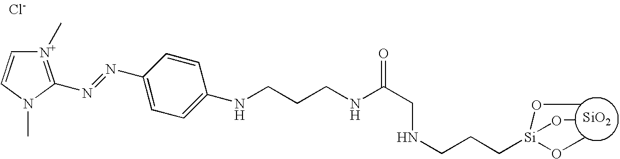

A mixture of aminopropyl-substituted nanoparticles obtainable according to Example A1 (0.4 g, 26.2% dispersion in ethanol), the chloroacetamide of formula (108) (200 mg), potassium carbonate (72 mg) and isopropanol (15 ml) is heated to 90° C. overnight. The mixture is then cooled to room temperature and the precipitate is filtered and washed with isopropanol, acetone then water. This gives the linked particles of the above formula as a red solid (189 mg). TEM shows monodisperse particles of average diameter of about 40 nm. TGA shows loss of 47.46%, corresponding to the organic material and elemental analysis gives C, 24.59; H, 3.68; N, 9.68%.

### Example A24

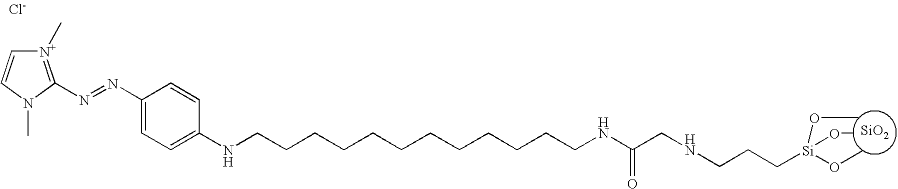

The above compound is prepared in analogy to Example A23 using the compound of formula (110). TGA shows loss of 41.74%, corresponding to the organic material. Elemental analysis shows C, 22.93; H, 3.05; N, 4.13. Upon further washing with water, elemental analysis shows C, 36.54; H, 4.89; N, 6.72%. TEM analysis shows uniform spheres of 30-40 nm diameter.

### Example A25

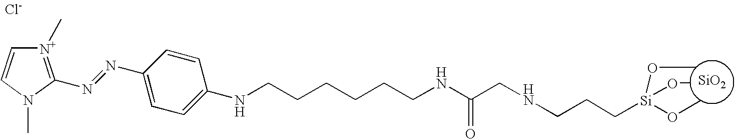

The above compound is prepared in analogy to Example A23 using the compound of formula (109). TGA shows loss of 38.44%, corresponding to the organic material. Elemental analysis shows C, 29.62; H, 4.22; N, 8.70%. TEM shows uniform spheres of 30-40 nm.

### EXAMPLE B

### Application

The washing fastness of the dyed hair is analyzed using the Gray scale according to: Industrial Organic Pigments, Herbst & Hunger, 2nd ed. engl. p. 61, no. 10: DIN 54001-8-1982, “Herstellung und Bewertung der Änderung der Farbe”, ISO 105-A02-1993.

### Example B1

50 mg of the functionalized particle obtainable according to the above Example A6 is dispersed in 50 g water. This red dyeing agent is applied on the dry hair (two blond, two middle blond and two damaged hair strands) and allowed to stand for 20 min. at room temperature. Then, the strands are rinsed under tap water and dried for 12 hours.

Washing fastness: 10× washed with shampoo.

### Results:

| Strand | Color Result | Washing Fastness |
|---|---|---|
| blond | Red/good | 4-5 |
| middleblond | Red/good | 3-4 |
| damaged | Red/good | 4 |

In the same way, application of the following dyes to hair gave the following results:

### Example B2

### Dye from Example A10

| Strand | Color Result | Washing Fastness |
|---|---|---|
| blond | Red/good | 3-4 |
| middleblond | Red/good | 3-4 |
| damaged | Red/good | 3 |

### Example B3

### Dye from Example A11

| Strand | Color Result | Washing Fastness |
|---|---|---|
| blond | Red/good | 3-4 |
| middleblond | Red/good | 3-4 |
| damaged | Red/good | 3 |

### Example B4

### Dye from Example A14

| Strand | Color Result | Washing Fastness |
|---|---|---|
| blond | Red/good | 3 |
| middleblond | Red/good | 3-4 |
| damaged | Red/good | 3 |

### Example B5

### Dye from Example A17

| Strand | Color Result | Washing Fastness |
|---|---|---|
| Blond | Red/good | 3-4 |
| middleblond | Red/good | 4 |
| damaged | Red/good | 3 |

### Example B6

### Dye from Example A21

| Strand | Color Result | Washing Fastness | dE |
|---|---|---|---|
| Blond | Red/good | 2-3 | 22.1 |
| middleblond | Red/good | 3 | 6.5 |
| damaged | Red/good | 3-4 | 9.1 |

### Example B7

### Dye from Example A20

| Strand | Color Result | Washing Fastness | dE |
|---|---|---|---|
| Blond | Red/good | 2-3 | 23.7 |
| middleblond | Red/good | 3 | 11.2 |
| damaged | Red/good | 3 | 13.3 |

### Example B8

### Dye from Example A22

| Strand | Color Result | Washing Fastness | dE |
|---|---|---|---|
| Blond | Red/good | 3 | 17.1 |
| middleblond | Red/good | 4 | 3.6 |
| damaged | Red/good | 3-4 | 11.4 |

1. A method for dyeing of keratin-containing fibers comprising treating the fibers with at least one functionalized particle comprising on the surface an organic chromophore which is bound via a bridge member, wherein
the functionalized particles are based on SiO₂, Al₂O₃ or mixtures thereof, and
the functionalized particles carry a positive charge.

2. A method according to claim 1, wherein the functionalized particles comprise, covalently bound to an oxygen atom on the surface, a radical of formula 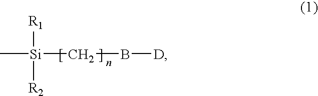
wherein
R₁ and R₂ are independently of each other hydrogen, particle surface-O—, or a substituent,
B is the direct bond or a bridge member,
D is a radical of an organic chromophore, and
n is 1, 2, 3, 4, 5, 6, 7, 8, 9, 10, 11 or 12.

3. A method according to claim 2, wherein
R₁ and R₂ independently of each other are hydrogen; C₁-C₂₅alkyl which may be interrupted by —O—, —S— or —N(R₃)—; C₂-C₂₄alkenyl; phenyl; C₇-C₉-phenylalkyl; —OR₅; 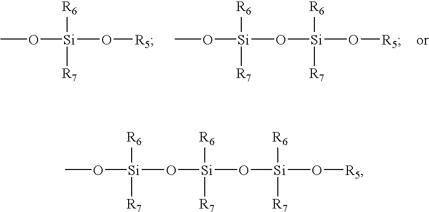
R₅ is hydrogen; C₁-C₂₅alkyl which may be interrupted by —O—, —S— or —N(R₃)—; C₂-C₂₄alkenyl; phenyl; C₇-C₉phenylalkyl; 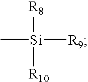
or the particle surface,
R₆ and R₇ independently of each other are hydrogen; C₁-C₂₅alkyl which may be interrupted by —O—, —S— or —N(R₃)—; C₂-C₂₄alkenyl; phenyl; C₇-C₉phenylalkyl; or —OR₅, and
R₈, R₉ and R₁₀ independently of each other are hydrogen; C₁-C₂₅alkyl which may be interrupted by —O—, —S— or —N(R₃)—; C₂-C₂₄alkenyl; phenyl; or C₇-C₉phenylalkyl, and wherein
R₃ is hydrogen, C₁-C₁₂alkyl or hydroxyl-substituted C₁-C₁₂alkyl.

4. A method according to claim 2, wherein
n is 1, 2, 3, 4, 5, 6, 7 or 8.

5. A method according to claim 2, wherein
B is the direct bond, —O—, —S—, —N(R₃)— or a bridge member of formula -A₁-C₁-C₂₅alkylene-A₂-, -A₁-C₁-C₂₅alkylene-phenylene-A₂- or -A₁-phenylene-C₁-C₂₅alkylene-A₂-, wherein
A₁ and A₂ are the direct bond, —O—, —S—, —N(R₃)—, —CO—, —O—CO—, —CO—O—, —N(R₃)—CO— or —CO—N(R₃)—,
the C₁-C₂₅alkylene radical is uninterrupted or interrupted by at least one of the radicals selected from the group consisting of —O—, —S—, —N(R₃)—, —N⁺(R₃)₂—, —CO—, —O—CO—, —CO—O—, —N(R₃)—CO—, —CO—N(R₃)— and phenylene, and
wherein R₃ is hydrogen, C₁-C₁₂alkyl or hydroxyl-substituted C₁-C₁₂alkyl.

6. A method according to claim 2, wherein
D is a radical selected from the group consisting of acridine, anthraquinone, azomethine, monoazo, disazo, polyazo, benzodifuranone, coumarin, diketopyrrolopyrrole, dioxazine, diphenylmethane, formazan, indigoid, methine, polymethine, naphthalimide, naphthoquinone, nitroaryl, oxazine, perinone, perylene, phenazine, phthalocyanine, pyrenequinone, quinacridone, quinoneimine, quinophthalone, stilbene, styryl, thiazine, thioxanthene, triarylmethane, xanthene and metal complex dye.

7. A method according to claim 6, wherein
D is a radical selected from the group consisting of anthraquinone, monoazo, azomethine, styryl, methine, polymethine, triarylmethane and metal complex dye.

8. A method according to claim 1, wherein the functionalized particles comprise in addition, covalently bound to an oxygen atom on the surface, a radical of the formula (11) 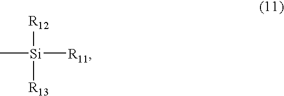
wherein
R₁₂ and R₁₃ are independently of each other hydrogen, particle surface-O—, or a substituent, R₁₁ is C₁-C₂₅alkyl or C₂-C₂₄alkenyl, each of which is unsubstituted or substituted by amino, mercapto, phenyl or hydroxyl and is uninterrupted or interrupted by —O—, —S—, —N(R₁₄)—, —CO—, —O—CO—, —CO—O—, —N(R₁₄)—CO—, —CO—N(R₁₄)— or phenylene; C₅-C₁₂cycloalkyl; C₅-C₁₂cycloalkenyl; or a polymerizable group or a polymer each of which may be bound via a bridge member, and
R₁₄ is hydrogen or unsubstituted or substituted C₁-C₁₂alkyl.

9. A method according to claim 8, wherein the functionalized particles comprise in addition, covalently bound to an oxygen atom on the surface, a radical of the formula (12) 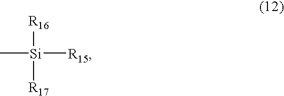
wherein
R₁₆ and R₁₇ are independently of each other hydrogen, particle surface-O—, or a substituent,
R₁₅ is C₁-C₂₅alkyl or C₂-C₂₄alkenyl, each of which is unsubstituted or substituted by amino, mercapto, phenyl or hydroxyl and is uninterrupted or interrupted by —O—, —S—, —N(R₁₈)—, —N⁺(R₁₈)₂—, —CO—, —O—CO—, —CO—O—, —N(R₁₈)—CO—, —CO—N(R₁₈)— or phenylene; C₅-C₁₂cycloalkyl; C₅-C₁₂cycloalkenyl; or a polymerizable group or a polymer each of which may be bound via a bridge member,
R₁₈ is hydrogen or unsubstituted or substituted C₁-C₁₂alkyl, and
wherein R₁₅ or R₁₈ additionally comprise a cationic group, preferably a cationic ammonium group.

10. A method according to claim 9, wherein the functionalized particles carry at least one cationic ammonium group of formula —N(R₁*)₃, wherein the three radicals R₁* can have the same or different meanings, and
R₁* is hydrogen; C₁-C₁₂alkyl which can be interrupted by —O— and can be substituted by hydroxyl or phenyl, and wherein the phenyl radical can be further substituted by C₁-C₈alkyl, C₁-C₈alkoxy or halogen; or phenyl which can be substituted by C₁-C₈alkyl, C₁-C₈alkoxy or halogen.

11. A method according to claim 10, wherein
R₁* is hydrogen or C₁-C₁₂alkyl.

12. A method according to claim 1, wherein the functionalized particles have a spherical shape.

13. A method according to claim 1, wherein the functionalized particles have a mean particle size of 1 to 1000 nm.

14. A method according to claim 13, wherein the functionalized particles have a mean particle size of 1 to 200 nm.

15. A method according to claim 1, wherein the functionalized nanoparticles are based on SiO₂.

16. A method according to claim 1, wherein the keratin-containing fibers are treated with at least one functionalized particle as defined in claim 1 and an oxidative agent and, optionally, a further direct dye.

17. A method according to claim 1, wherein the keratin fibers are treated with at least one functionalized particle as defined in claim 1 and at least one oxidative dye, or treating the keratin-containing fibers with at least one functionalized particle as defined in claim 1 and at least one oxidative dye and an oxidative agent.

18. A method according to claim 1 wherein the keratin-containing fiber is human hair.

19. Functionalized particles comprising, covalently bound to an oxygen atom on the surface, a radical of formula 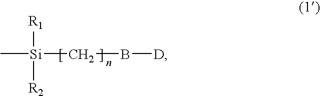
wherein
the particles are based on SiO₂, Al₂O₃ or mixtures thereof,
the functionalized particles carry a positive charge,
R₁ and R₂ are independently of each other hydrogen, particle surface-O—, or a substituent,
B is the direct bond or a bridge member,
n is 1, 2, 3, 4, 5, 6, 7, 8, 9, 10, 11 or 12, and
D is a radical of formula 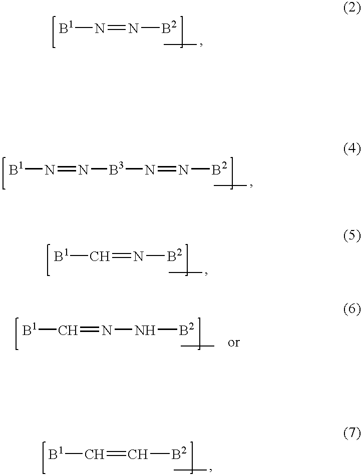
wherein
B³ is unsubstituted or substituted phenylene or naphthylene, and
B¹ and B², independently of each other, are optionally substituted phenyl, naphthyl or a heterocylic group of formula 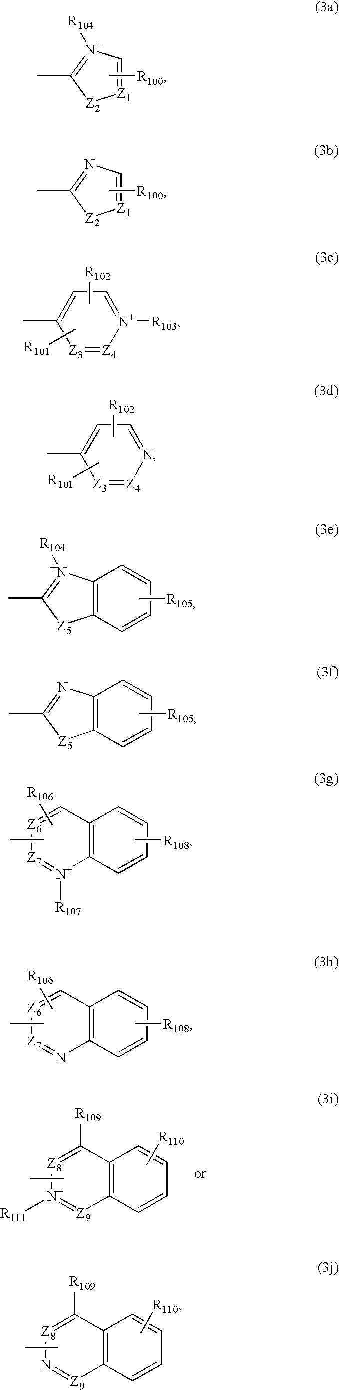
wherein
Z₂ and Z₅ are —O—; —S—; or a radical NR₁₁₂,
Z₁, Z₃, Z₄, Z₆, Z₇, Z₈ and Z₉ are independently from each other N or a radical CR₁₁₃;
R₁₀₀, R₁₀₁, R₁₀₂, R₁₀₅, R₁₀₆, R₁₀₈, R₁₀₉, R₁₁₀ and R₁₁₃ are independently from each other hydrogen; halogen; hydroxy; unsubstituted or substituted C₁-C₁₂alkyl; unsubstituted or substituted phenyl; nitrile; C₂-C₄alkanoylamino; carbamoyl; ureido; sulfonylamino;
C₁-C₁₂alkylthio; or a radical of formula —N(R₁₁₄)R₁₁₅, —N(R₁₁₄)(R₁₁₅)R₁₁₆ or —OR₁₁₄;
R₁₀₃, R₁₀₄, R₁₀₇, R₁₁₁ and R₁₁₂ are independently from each other hydrogen; unsubstituted or substituted C₁-C₁₂alkyl; or unsubstituted or substituted phenyl; and
R₁₁₄, R₁₁₅ and R₁₁₆ are independently from each other hydrogen; unsubstituted or substituted C₁-C₁₂alkyl; or unsubstituted or substituted triazinyl or phenyl.

20. Functionalized particles according to claim 19, wherein
at least one of the radicals B¹ and B² is a heterocyclic group selected from formulae (3a) to (3j).

21. Functionalized particles according to claim 19, wherein R₁₀₃, R₁₀₄, R₁₀₇, R₁₁₁ and R₁₁₂ are C₁-C₁₂alkyl, preferably C₁-C₄alkyl.

22. A method according to claim 4, wherein n is 3.

23. A method according to claim 9, wherein
the functionalized particles comprise in addition, covalently bound to an oxygen atom on the surface, a radical of the formula (12) 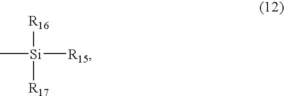
wherein
R₁₆ and R₁₇ are independently of each other hydrogen, particle surface-O—, or a substituent,
R₁₅ is C₁-C₂₅alkyl or C₂-C₂₄alkenyl, each of which is unsubstituted or substituted by amino, mercapto, phenyl or hydroxyl and is uninterrupted or interrupted by —O—, —S—, —N(R₁₈)—, —N⁺(R₁₈)₂—, —CO—, —O—CO—, —CO—O—, —N(R₁₈)—CO—, —CO—N(R₁₈)— or phenylene; C₅-C₁₂cycloalkyl; C₅-C₁₂cycloalkenyl; or a polymerizable group or a polymer each of which may be bound via a bridge member,
R₁₈ is hydrogen or unsubstituted or substituted C₁-C₁₂alkyl, and
wherein R₁₅ or R₁₈ additionally comprise a cationic ammonium group.

24. A method according to claim 13, wherein the functionalized particles have a mean particle size of 1 to 600 nm.

25. A method according to claim 14, wherein the functionalized particles have a mean particle size of 1 to 100 nm.
